(19) United States
(12) Patent Application Publication
(10) Pub. No.: US 2022/0218762 A1
(43) Pub. Date: Jul. 14, 2022

(54) COMPOSITIONS AND METHODS FOR MODULATING COGNITIVE BEHAVIOR

(71) Applicant: The Regents of The University of California, Oakland, CA (US)

(72) Inventors: Elaine Y. Hsiao, Los Angeles, CA (US); Christine Olson, Los Angeles, CA (US); Alonso Iniguez, Los Angeles, CA (US)

(21) Appl. No.: 17/615,297

(22) PCT Filed: Jun. 1, 2020
(86) PCT No.: PCT/US20/35539

§ 371 Date: Nov. 30, 2021

Related U.S. Application Data
(60) Provisional application No. 62/855,290, filed on May 31, 2019.

(51) Int. Cl. A61K 35/74; A61P 25/28; C07K 16/24

(52) U.S. Cl. CPC A61K 35/74; C07K 16/244; A61P 25/28

(57) ABSTRACT

The present disclosure provides methods of treating hypoxia-induced cognitive impairment. Also disclosed are microbiome modulators, such as ketogenic-diet-suppressed bacterial species or antibiotics effective against a ketogenic-diet-boosted bacterial species, for use in treatment of cognitive impairment. The disclosure also provides methods of selecting a subject having hypoxia-induced cognitive impairment and methods of obtaining a prognostic indicator of hypoxia-induced cognitive impairment in a subject who receives a dosage of a microbiome modulator.

### RELATED APPLICATION

This application claims a right of priority to and the benefit of the filing date of U.S. Provisional Application No. 62/855,290, filed on May 31, 2019, which is hereby incorporated by reference in its entirety.

### STATEMENT OF RIGHTS

This invention was made with government support under Grant Number W911NF-17-1-0402, awarded by the U.S. Army, Army Research Office. The government has certain rights in the invention.

### BACKGROUND

Cognitive impairment, characterized by deficient attention, causal reasoning, as well as learning and memory, is a pressing public health concern that afflicts 16 million Americans and is increasing globally. Genetic, environmental, and behavioral factors disrupt cognition, for which many biological pathways are implicated, including neuroinflammation, mitochondrial dysfunction, and blood-brain-barrier integrity.

A primary environmental risk factor for cognitive impairment is hypoxia, which can occur, for example, in response to high altitude, sleep apnea, or ischemia. Therefore, one particular form of cognitive impairment is hypoxia-induced cognitive impairment.

Despite the importance of the issue, exactly how environmental factors, including diet, exercise, and socioeconomic status, lead to cognitive impairment (e.g., hypoxia-induced cognitive impairment) remains poorly understood. In addition, clinically tractable interventions to ameliorate cognitive impairment are lacking.

### SUMMARY OF THE INVENTION

In some aspects, methods of treating cognitive impairment, particularly hypoxia-induced cognitive impairment, in a subject include administering an effective amount of a microbiome modulator to the subject. The microbiome modulator can be a ketogenic-diet-suppressed bacterial species such as Clostridium cocleatum, an antibiotic effective against a ketogenic-diet-boosted bacterial species such as Bilophila wadsworthia, or a combination of such microbiome modulators.

In other aspects, methods of selecting a subject who has hypoxia-induced cognitive impairment include obtaining a level for a biomarker associated with hypoxia-induced cognitive impairment from a sample of a subject and selecting the subject if the level differs from a control level by more than a threshold. In additional aspects, methods of obtaining a prognostic indicator of hypoxia-induced cognitive impairment in a subject include obtaining a level for a biomarker associated with hypoxia-induced cognitive impairment from a sample of a subject who has received a dose of a microbiome modulator, determining that the level differs by more than a differential from a reference level, and determining that the hypoxia-induced cognitive impairment is improving if the differential is less than a predetermined differential. The reference level can be representative of a subject who does not have hypoxia-induced cognitive impairment or can be representative of the subject having hypoxia-induced cognitive impairment before administration of the microbiome modulator.

In certain aspects, microbiome modulators for use in treatment of hypoxia-induced cognitive impairment in a subject are disclosed. In some embodiments, the microbiome modulator includes a ketogenic-diet-suppressed bacterial species (e.g., Clostridium cocleatum). In some embodiments, the microbiome modulator comprises an antimicrobial agent active against a ketogenic-diet-boosted bacterial species (e.g., Bilophila wadsworthia, in which case the antibiotic may be imipenem, cefoxitin, or ticarcillin). In some embodiments, the microbiome modulator includes both a ketogenic-diet-suppressed bacterial species (e.g., Clostridium cocleatum) and such an antimicrobial agent active against a ketogenic-diet-boosted bacterial species.

In some embodiments, the microbiome modulator used/administered is a bacterial species from the genus Clostridium, or an antibiotic (or another agent) effective against a ketogenic-diet-boosted bacterial species from the genus Bilophila, or a combination thereof.

In some aspects, methods of treating hypoxia-induced cognitive impairment in a subject include administering an effective amount of an anti-IL-12p40 agent to the subject. In some embodiments, the anti-IL-12p40 agent includes an anti-IL-12p40 antibody or an antigen-biding fragment thereof.

In various embodiments, the biomarkers associated with hypoxia-induced cognitive impairment can be one or more of the following: Actb, Atg2a, Atp5d, Atp6v0e2, Camkv, Cldn11, Cldn5, Dctn4, Erbb3, Gabarap, Mag, Mapk11, Mbp, Micall1, Mobp, Nfasc, Nipa14, Pik3r2, Scn1b, Tubd1, Zfpm1, Adam7, Adcyap1, Adig, Adipoq, Adora2a, Adrb3, Aoc3, Avp, Baiap3, C3, Calb2, Car3, Cartpt, Cbin1, Cdo1, Ceacam10, Cidec, Cwc22, Defb20, Defb48, Dio2, Drd1, Ecel1, Etnppl, Fabp4, Fgf12, Fggy, Flvcr2, G0s2, Gad2, Glra1, Glra3, Gm42743, Gm44862, Gpx5, Hp, Klhl1, Lcn8, Lgr5, Lyzf1, Marcks, mCG_18947, Meis1, Myo19, Pbx3, Penk, Plin1, Plin4, Pmch, Pnpla2, Prdm1, Retn, Retnla, Rgs9, Rrad, Rspo1, Scd1, Spink3, Spink1, Sslp1, Svs1, Svs4, Svs5, Svs6, Tacr1, and Wisp1.

### BRIEF DESCRIPTION OF THE FIGURES

FIG. 1A to FIG. 1L: The Ketogenic Diet Potentiates Hypoxia-Induced Impairments in Cognitive Behavior. FIG. 1A) Experimental timeline: Conventionally-colonized (specific pathogen free, SPF) mice were fed a control diet (CD) for 7 days prior to intermittent hypoxia (Hyp) or normoxia (Mock) exposure for 5 days, followed by Barnes maze testing 4 days later. FIG. 1B) Representative Barnes maze traces for SPF mice fed the CD and exposed to Mock or Hyp. White lines indicate movement trajectories, whereas blue hues denote increasing durations of time spent at a specific location. Orange circles indicate the escape hole. FIG. 1C) Latency to enter the escape hole of the Barnes maze across six 300-second trials for SPF mice fed the CD and exposed to Mock or Hyp. (Two-way ANOVA with Sidak, n=13-17). FIG. 1D) Errors made as measured by number of incorrect nose pokes across six Barnes maze trials for SPF mice fed the CD and exposed to Mock or Hyp. (Two-way ANOVA with Sidak, n=13-17). FIG. 1E) Errors made during the final Barnes maze trial (probe) for SPF mice fed the CD and exposed to Mock or Hyp. (Unpaired two-tailed Students t-test, n=13-7). FIG. 1F) Search strategy used during the probe trial for SPF mice fed the CD and exposed to Mock or Hyp. (n=13-17). FIG. 1G) Experimental timeline: SPF mice were fed a ketogenic diet (KD) for 7 days prior to Hyp or Mock exposure for 5 days, followed by Barnes maze testing 4 days later. (n=11-13). FIG. 1H) Representative Barnes maze traces for SPF mice fed the KD and exposed to Mock or Hyp. White lines indicate movement trajectories, whereas blue hues denote increasing durations of time spent at a particular location. Orange circles denote the escape hole. FIG. 1I) Latency to enter the escape hole across six 300-second trials for SPF mice fed the KD and exposed to Mock or Hyp. (Two-way ANOVA with Sidak, n=11-13). FIG. 1J) Errors made as measured by number of incorrect nose pokes across six Barnes maze trials for SPF mice fed the KD and exposed to Mock or Hyp. (Two-way ANOVA with Sidak, n=11-13). FIG. 1K) Errors made during the probe trial for SPF mice fed the KD and exposed to Mock or Hyp. (Unpaired two-tailed Students t-test, n=11-13). FIG. 1L) Search strategy used during the probe trial for SPF mice fed the CD and exposed to Mock or Hyp. (n=11-13). Data are presented as mean±S.E.M. *p<0.05, **p<0.01, ***p<0.001, ****p<0.0001. n.s.=not statistically significant. SPF=specific pathogen-free (conventionally-colonized), CD=control diet, KD=ketogenic diet, Mock=intermittent normoxia exposure, Hyp=intermittent hypoxia exposure.

FIG. 2A to FIG. 2J: There is no significant effect of hypoxia or ketogenic diet on locomotion in the Barnes maze. FIG. 2A) Average velocity of locomotion across trials in the Barnes maze for SPF mice fed CD and exposed to Mock or Hyp. (Two-way ANOVA with Sidak, n=12-13). FIG. 2B) Total distance travelled across trials in the Barnes maze for SPF mice fed CD and exposed to Mock or Hyp. (Two-way ANOVA with Sidak, n=12-13). FIG. 2C) Average velocity of locomotion across trials in the Barnes maze for SPF mice fed KD and exposed to Mock or Hyp. (Two-way ANOVA with Sidak, n=11-13). FIG. 2D) Total distance travelled across trials in the Barnes maze for male SPF mice fed KD and exposed to Mock or Hyp. (Two-way ANOVA with Sidak, n=11-13). FIG. 2E) Average velocity of locomotion across trials in the Barnes maze for SPF mice pre-treated with Abx, fed KD and exposed to Mock or Hyp. (Two-way ANOVA with Sidak, n=8). FIG. 2F) Total distance travelled across trials in the Barnes maze for SPF mice pre-treated with Abx, fed KD and exposed to Mock or Hyp. (Two-way ANOVA with Sidak, n=8). FIG. 2G) Average velocity of locomotion across trials in the Barnes maze for GF mice transplanted with SPF KD Mock or SPF KD Hyp microbiota. (Two-way ANOVA with Sidak, n=8). FIG. 211) Total distance travelled across trials in the Barnes maze for GF mice transplanted with SPF KD Mock or SPF KD Hyp microbiota. (Two-way ANOVA with Sidak, n=8). FIG. 2I) Average velocity of locomotion across trials in the Barnes maze for GF mice monocolonized with Clos or Bilo. (Two-way ANOVA with Sidak, n=14-15). FIG. 2J) Total distance travelled across trials in the Barnes maze for GF mice monocolonized with Clos or Bilo. (Two-way ANOVA with Sidak, n=14-15). Data are presented as mean±S.E.M. *p<0.05, **p<0.01, ***p<0.001. n.s.=not statistically significant. Abx=treated with antibiotics (ampicillin, vancomycin, metronidazole, neomycin), KD=ketogenic diet, Mock=intermittent normoxia exposure, Hyp=intermittent hypoxia exposure, GF=germ-free, GF+Mock=GF mice transplanted with microbiota from SPF mice fed KD and exposed to Mock, GF+Hyp=GF mice transplanted with microbiota from SPF mice fed KD and exposed to Hyp, GF+Clos=GF mice monocolonized with C. cocleatum. GF+Bilo=GF mice monocolonized with B. wadsworthia. Data are presented as mean±S.E.M. *p<0.05. n.s.=not statistically significant. SPF=specific pathogen-free (conventionally-colonized), CD=control diet, KD=ketogenic diet, Mock=intermittent normoxia exposure, Hyp=intermittent hypoxia exposure.

FIG. 3A to FIG. 3E: There is no significant effect of hypoxia or ketogenic diet on behavior of male mice in open field exploration and prepulse inhibition tasks. FIG. 3A) Time spent in the center arena during the open field task for SPF mice fed CD or KD and exposed to Mock or Hyp. (One-way ANOVA with Sidak, n=6). FIG. 3B) Number of entries into the center arena during the open field task for SPF mice fed CD or KD and exposed to Mock of Hyp. (One-way ANOVA with Sidak, n=6). FIG. 3C) Total distance travelled during the open field task for SPF mice fed CD or KD and exposed to Mock of Hyp. (One-way ANOVA with Sidak, n=6). FIG. 3D) Average velocity of locomotion during the open field task for SPF mice fed CD or KD and exposed to Mock of Hyp. (One-way ANOVA with Sidak, n=6). FIG. 3E) Percent pre-pulse inhibition (PPI) in response to a 5, 10, or 15 dB pre-pulse in SPF mice fed CD or KD and exposed to Mock of Hyp. (Two-way ANOVA with Sidak, n=4-8, negative dots out of range). Data are presented as mean±S.E.M. n.s.=not statistically significant. SPF=specific pathogen-free (conventionally-colonized), CD=control diet, KD=ketogenic diet, Mock=intermittent normoxia exposure, Hyp=intermittent hypoxia exposure.

FIG. 4A to FIG. 4C: The ketogenic diet significantly potentiates hypoxia-induced impairments in cognitive behavior in the Barnes maze. FIG. 4A) Latency to enter the escape hole of the Barnes maze across six 300-second trials for SPF mice fed the CD and exposed to Mock or Hyp. (Two-way ANOVA with Sidak, n=11-17). FIG. 4B) Errors made as measured by number of incorrect nose pokes across six Barnes maze trials for SPF mice fed the CD and exposed to Mock or Hyp. (Two-way ANOVA with Sidak, n=11-17). FIG. 4C) Search strategy used during the probe trial for SPF mice fed the CD and exposed to Mock or Hyp. (n=11-17). Data are as displayed in FIG. 1. Data are presented as mean±S.E.M. *p<0.05, **p<0.01. SPF=specific pathogen-free (conventionally-colonized), CD=control diet, KD=ketogenic diet, Mock=intermittent normoxia exposure, Hyp=intermittent hypoxia exposure.

FIG. 5A to FIG. 5L: Alterations in the Gut Microbiota Contribute to Ketogenic Diet and Hypoxia-Induced Impairments in Cognitive Behavior. FIG. 5A) Experimental timeline: Conventionally-colonized (specific pathogen free, SPF) mice were pre-treated with oral antibiotics (Abx) for 7 days prior to feeding with a ketogenic diet (KD), exposure to intermittent hypoxia (Hyp) or normoxia (Mock), and Barnes maze testing. FIG. 5B) Representative Barnes maze traces for SPF or Abx mice fed the KD and exposed to Hyp. White lines indicate movement trajectories, whereas blue hues denote increasing durations of time spent at a particular location. Orange circles indicate the escape hole. FIG. 5C) Latency to enter the escape hole of the Barnes maze across six 300-second trials for SPF or Abx mice fed the KD and exposed to Mock or Hyp. (Two-way ANOVA with Sidak, n=8 for Abx groups; SPF data are as in FIG. 1). FIG. 5D) Errors made as measured by number of incorrect nose pokes across six Barnes maze trials for SPF or Abx mice fed the KD and exposed to Mock or Hyp. (Two-way ANOVA with Sidak, n=8 for Abx groups; SPF data are as in FIG. 1). FIG. 5E) Errors made during the probe trial of the Barnes maze for SPF or Abx mice fed the KD and exposed to Mock or Hyp. (One-way ANOVA with Dunnett, n=8 for Abx groups; SPF data are as in FIG. 1). FIG. 5F) Search strategy used during probe trial of the Barnes maze for SPF or Abx mice fed the KD and exposed to Mock or Hyp. (n=8). FIG. 5G) Experimental timeline: GF mice were gavaged with fecal microbiota from SPF KD Mock or SPF KD Hyp donors (from FIG. 1) and subjected to Barnes maze testing 4 days later. FIG. 5H) Representative Barnes maze traces for GF transplanted with fecal microbiota from SPF KD Mock or SPF KD Hyp donors. Transplanted recipient mice receiving SPF KD Mock microbiota are denoted GF+Mock. Transplanted recipient mice receiving SPF KD Hyp microbiota are denoted GF+Hyp. White lines indicate movement trajectories, whereas blue hues denote increasing durations of time spent at a particular location. Orange circles denote the escape hole. FIG. 5I) Latency to enter the escape hole across six 300-second trials for GF mice transplanted with SPF KD Mock or SPF KD Hyp microbiota. (Two-way ANOVA with Sidak, n=14-15). FIG. 5J) Errors made during as measured by number of incorrect nose pokes across six Barnes maze trials for GF mice transplanted with SPF KD Mock or SPF KD Hyp microbiota. (Two-way ANOVA with Sidak, n=14-15). FIG. 5K) Errors made during the probe trial of the Barnes maze for GF mice transplanted with SPF KD Mock or SPF KD Hyp microbiota. (Unpaired two-tailed Students t-test, n=14-15). FIG. 5L) Search strategy used during probe trial of the Barnes maze for GF mice transplanted with SPF KD Mock or SPF KD Hyp microbiota. (n=14-15). Data are presented as mean±S.E.M. *p<0.05, **p<0.01, ***p<0.001, ****P<0.0001. n.s.=not statistically significant. SPF=specific pathogen-free (conventionally-colonized), Abx=treated with antibiotics (ampicillin, vancomycin, metronidazole, neomycin), KD=ketogenic diet, Mock=intermittent normoxia exposure, Hyp=intermittent hypoxia exposure, GF=germ-free, GF+Mock=GF mice transplanted with microbiota from SPF mice fed KD and exposed to Mock, GF+Hyp=GF mice transplanted with microbiota from SPF mice fed KD and exposed to Hyp.

FIG. 6A to FIG. 611: Germ-free mice fed the ketogenic diet are resistant to hypoxia-induced impairment in cognitive behavior. FIG. 6A) Experimental timeline: GF mice were fed KD for 7 days prior to Hyp or Mock exposure for 5 days, followed by Barnes maze testing 4 days later. FIG. 6B) Representative Barnes maze traces for GF mice fed the KD and exposed to Mock or Hyp. White lines indicate movement trajectories, whereas blue hues denote increasing durations of time spent at a particular location. Orange circles indicate the escape hole. FIG. 6C) Latency to enter the escape hole of the Barnes maze across six 300-second trials for male GF mice fed the KD and exposed to Mock or Hyp. (Two-way ANOVA with Sidak, n=14-15 for GF groups; SPF data are as in FIG. 1). FIG. 6D) Errors made as measured by number of incorrect nose pokes across six Barnes maze trials for GF mice fed the KD and exposed to Mock or Hyp. (Two-way ANOVA with Sidak, n=14-15 for GF groups; SPF data are as in FIG. 1). FIG. 6E) Errors made during the probe trial of the Barnes maze for GF mice fed the KD and exposed to Mock or Hyp. (One-way ANOVA with Sidak, n=14-15 for GF groups; SPF data are as in FIG. 1). FIG. 6F) Search strategy used during probe trial of the Barnes maze for GF mice fed the KD and exposed to Mock or Hyp. (n=14-15). FIG. 6G) Average velocity of locomotion across trials in the Barnes maze for GF mice fed KD and exposed to Mock or Hyp. (Two-way ANOVA with Sidak, n=14-15). FIG. 611) Total distance travelled across trials in the Barnes maze for GF mice fed KD and exposed to Mock or Hyp. (Two-way ANOVA with Sidak, n=14-15). Data are presented as mean±S.E.M. n.s.=not statistically significant. SPF=specific pathogen-free (conventionally-colonized), GF=germ-free, KD=ketogenic diet, Mock=intermittent normoxia exposure, Hyp=intermittent hypoxia exposure.

FIG. 7A to FIG. 7M: Bilophila is Enriched by the Ketogenic Diet and Hypoxia, and Sufficiently Impairs Cognitive Behavior. FIG. 7A) Representative principal coordinates analysis of weighted UniFrac distance based on 16S rRNA gene profiling of feces from SPF mice fed KD and exposed to Hyp or Mock. (n=4 cages). FIG. 7B) Average taxonomic distributions of low abundance bacteria from 16S rRNA gene sequencing data of feces from SPF mice fed KD and exposed to Hyp or Mock. (n=13 cages). FIG. 7C) Relative abundances of Clostridium cocleatum (left) and Bilophila spp. (right) in fecal microbiota of SPF mice fed KD and exposed to Hyp or Mock. (Kruskal wallis with Bonferroni, n=13 cages). FIG. 7D) Representative principal coordinates analysis of weighted UniFrac distance based on 16S rRNA gene profiling of feces from GF mice transplanted with fecal microbiota from SPF KD Mock or SPF KD Hyp mice (in panels A-C). (n=4-5 cages) FIG. 7E) Average taxonomic distributions of low abundance bacteria from 16S rRNA gene sequencing data of feces from GF mice transplanted with microbiota from SPF KD Mock or SPF KD Hyp mice (n=9 cages). FIG. 7F) Relative abundances of Clostridium cocleatum (left) and Bilophila spp. (right) in fecal microbiota of GF mice transplanted with microbiota from SPF KD Mock or SPF KD Hyp mice. (Kruskal wallis with Bonferroni, n=9 cages). FIG. 7G) Experimental timeline: GF mice were gavaged with cultured Clostridium cocleatum (Clos) or Bilophila wadsworthia (Bilo) and subjected to Barnes maze testing 4 days later. FIG. 7H) Representative Barnes maze traces for GF mice monocolonized with Clos or Bilo. White lines indicate movement trajectories, whereas blue hues denote increasing durations of time spent at a particular location. Orange circles denote the escape hole. FIG. 7I) Latency to enter the escape hole across six 300-second trials for GF mice monocolonized with Clos or Bilo. (Two-way ANOVA with Sidak, n=15, 24). FIG. 7J) Errors made as measured by number of incorrect nose pokes across six Barnes maze trials for GF mice monocolonized with Clos or Bilo. (Two-way ANOVA with Sidak, n=15, 24). FIG. 7K) Errors made during the probe trial of the Barnes maze for GF mice monocolonized with Clos or Bilo. (Unpaired two-tailed Students t-test, n=15, 24). FIG. 7L) Search strategy used during probe trial of the Barnes maze for GF mice monocolonized with Clos or Bilo. (n=15, 24). FIG. 7M) Effect size of hypoxia on latency to enter the escape hole during the probe trial, as measured by the difference between Hyp groups and respective Mock controls for SPF, Abx, GF, microbiota-transplanted (GF+Hyp-Mock), or monocolonized (GF+B-C) mice fed CD or KD. (Two-way ANOVA with Dunnett, n=8-24). Data are presented as mean±S.E.M. *p<0.05, **p<0.01, ***p<0.001. n.s.=not statistically significant. SPF=specific pathogen-free (conventionally-colonized), KD=ketogenic diet, Mock=intermittent normoxia exposure, Hyp=intermittent hypoxia exposure, GF=germ-free, GF+Mock=GF mice transplanted with SPF KD Mock microbiota, GF+Hyp=GF mice transplanted with SPF KD Hyp microbiota, GF+Clos=GF mice monocolonized with C. cocleatum. GF+Bilo=GF mice monocolonized with B. wadsworthia, Abx=treated with antibiotics (ampicillin, vancomycin, metronidazole, neomycin), CD=control diet.

FIG. 8A to FIG. 8D: There is no effect of the ketogenic diet and hypoxia on alpha diversity of the fecal microbiota. FIG. 8A) Number of observed operational taxonomic units (OTUs) in the gut microbiota, as measured by 16S rRNA gene sequencing data of feces from SPF mice fed the KD and exposed to Mock or Hyp. (n=8 cages). FIG. 8B) Average taxonomic distributions of abundant fecal bacteria from 16S rRNA gene sequencing of feces from SPF mice fed the KD and exposed to Mock or Hyp. (n=8 cages). FIG. 8C) Number of observed OTUs in the gut microbiota, as measured by 16S rRNA gene sequencing data of feces from GF mice transplanted with microbiota from SPF mice fed the KD and exposed to Mock or Hyp. (n=10-11 cages). FIG. 8D) Average taxonomic distributions of fecal bacteria from 16S rRNA gene sequencing of feces from GF mice transplanted with microbiota from SPF mice fed the KD and exposed to Mock or Hyp. (n=10-11 cages). SPF=specific pathogen-free (conventionally-colonized), KD=ketogenic diet, Mock=intermittent normoxia exposure, Hyp=intermittent hypoxia exposure, GF=germ-free, GF+Mock=GF mice transplanted with microbiota from SPF mice fed KD and exposed to Mock, GF+Hyp=GF mice transplanted with microbiota from SPF mice fed KD and exposed to Hyp.

FIG. 9A to FIG. 9C: The relative abundance of Bilophila in the gut microbiota correlates with cognitive impairment in the Barnes maze. FIG. 9A) Digital PCR for absolute quantification of Bilophila 16S copies per gram of fecal matter for SPF KD Mock, SPF KD Hyp, SPF CD Mock, SPF CD Hyp (n=4-5). FIG. 9B) Correlation of C. cocleatum and Bilophila spp. with latency to enter the escape hole during the probe trial of the Barnes maze for native and transplant groups. (Simple linear regression, n=17). FIG. 9C) Correlation of C. cocleatum and Bilophila spp. with number of errors made during the probe trial of the Barnes maze for native and transplant groups. (Simple linear regression, n=17).

FIG. 10A to FIG. 10K: Bilophila Colonization Phenocopies Ketogenic Diet and Hypoxia-Induced Impairments in Hippocampal Activity. FIG. 10A) Hippocampal long-term potentiation (LTP) as indicated by fEPSP slope in response to 100 Hz tetanus, expressed as a percentage of 20-minute baseline signal, from slice electrophysiology of brains from SPF mice fed KD and exposed to Hyp or Mock. (n=7-8). FIG. 10B) Average fEPSP slope during the last 5 minutes of hippocampal LTP recording for SPF mice fed KD and exposed to Hyp or Mock. (Unpaired two-tailed Students t-test, n=7-8). FIG. 10C) Hippocampal excitation to inhibition ratios extrapolated from fiber volley amplitude versus fEPSP slope from slice electrophysiology of brains from SPF mice fed KD and exposed to Hyp or Mock. (Two-way ANOVA with Sidak, n=7-8). Representative traces (inset). FIG. 10D) Hippocampal paired pulse facilitation from slice electrophysiology of brains from SPF mice fed KD and exposed to Hyp or Mock. (Two-way ANOVA with Sidak, n=7-8). Representative traces (inset). FIG. 10E) Hippocampal LTP as indicated by fEPSP slope in response to 100 Hz tetanus, expressed as a percentage of 20-minute baseline signal from slice electrophysiology of brains from GF mice monocolonized with Clos or Bilo. (n=12). FIG. 10F) Average fEPSP slope during the last 5 minutes of hippocampal LTP recording for GF mice monocolonized with Clos or Bilo. (Unpaired two-tailed Students t-test, n=12). FIG. 10G) Hippocampal excitation to inhibition ratios extrapolated from fiber volley amplitude versus fEPSP slope from slice electrophysiology of brains from GF mice monocolonized with Clos or Bilo. (Two-way ANOVA with Sidak, n=12). Representative traces (inset). FIG. 1011) Hippocampal paired pulse facilitation from slice electrophysiology of brains from GF mice monocolonized with Clos or Bilo. (Two-way ANOVA with Sidak, n=12). Representative traces (inset). FIG. 10I) Principal components analysis of all differentially regulated genes from RNA sequencing of CA3 subfields of the hippocampus from GF mice monocolonized with Clostridium cocleatum (Clos) or Bilophila wadsworthia (Bilo). (n=6). FIG. 10J) Volcano plot labeling genes with high fold change of differential expression in hippocampal CA3 from GF mice monocolonized with Bilo relative to Clos-monocolonized controls. (Wald test, n=6). FIG. 10K) Representative image of doublecortin (DCX)-positive neurons in the dentate gyrus of GF mice monocolonized with Clos or Bilo (left). Quantitation of DCX density per area of the dentate gyrus of GF mice monocolonized with Clos or Bilo (right). (Unpaired two-tailed Students t-test, n=4-5). Data are presented as mean±S.E.M. *p<0.05, **p<0.01, ***p<0.001. n.s.=not statistically significant. SPF=specific pathogen-free (conventionally-colonized), Abx=treated with antibiotics (ampicillin, vancomycin, metronidazole, neomycin), GF=germ-free, KD=ketogenic diet, Hyp=intermittent hypoxia exposure, Mock=intermittent normoxia exposure, GF+Clos=GF mice monocolonized with C. cocleatum. GF+Bilo=GF mice monocolonized with B. wadsworthia, DCX=doublecortin, LTP=long-term-potentiation, fEPSP=field excitatory post-synaptic potential.

FIG. 11A to FIG. 11D: Microbiota depletion diminishes ketogenic diet and hypoxia-induced impairments in hippocampal physiology. FIG. 11A) Hippocampal long-term potentiation (LTP) as indicated by fEPSP slope in response to 100 Hz tetanus, expressed as a percentage of 20-minute baseline signal from slice electrophysiology of brains from SPF or Abx mice fed KD and exposed to Hyp or Mock. Data for SPF groups are as in FIG. 4. (n=5-8). FIG. 11B) Average fEPSP slope during the last 5 minutes of hippocampal LTP recording for SPF or Abx mice fed KD and exposed to Hyp or Mock. Data for SPF groups are as in FIG. 4. (One-way ANOVA with Sidak, n=5-8). FIG. 11C) Hippocampal excitation to inhibition ratios extrapolated from fiber volley amplitude versus fEPSP slope from slice electrophysiology of brains from SPF or Abx mice fed KD and exposed to Hyp or Mock. Data for SPF groups are as in FIG. 4. (Two-way ANOVA with Sidak, n=5-8). Representative traces (inset). FIG. 11D) Hippocampal paired pulse facilitation from slice electrophysiology of brains from SPF or Abx mice fed KD and exposed to Hyp or Mock. Data for SPF groups are as in FIG. 4. (Two-way ANOVA with Sidak, n=5-8). Representative traces (inset). Data are presented as mean±S.E.M. *p<0.05, **p<0.01. n.s.=not statistically significant. SPF=specific pathogen-free (conventionally-colonized), Abx=treated with antibiotics (ampicillin, vancomycin, metronidazole, neomycin), GF=germ-free, KD=ketogenic diet, Hyp=intermittent hypoxia exposure, Mock=intermittent normoxia exposure, LTP=long-term-potentiation, fEPSP=field excitatory post-synaptic potential.

FIG. 12A to FIG. 12F: Microbiota depletion alters hippocampal gene expression in mice fed ketogenic diet and exposed to hypoxia. FIG. 12A) Differentially expressed genes (q<0.05) in the CA3 of the hippocampus in Abx and GF mice relative to SPF controls, all fed KD and exposed to Hyp. Bolded numbers are the total number of genes differentially regulated by each treatment. Of these, upregulated genes are displayed in green, whereas downregulated genes are displayed in magenta. Numbers in overlapping region denote the genes similarly altered by both Abx treatment and GF rearing. (Wald test, n=6). FIG. 12B) Principal components analysis of all differentially regulated genes from RNA sequencing of CA3 subfields of the hippocampus from SPF, GF or Abx-treated mice fed KD and exposed to Hyp (n=6). FIG. 12C) Heatmap of the 137 genes differentially regulated (q<0.05) in hippocampal CA3 of both GF and Abx-treated mice relative to SPF controls fed KD and exposed to Hyp. (Wald test, n=6). FIG. 12D) Top 10 pathways from GO-Term enrichment analysis of the 53 commonly upregulated genes (top, green) and 84 commonly downregulated genes (bottom, magenta) in hippocampal CA3 from GF and Abx-treated mice relative to SPF controls, all fed KD and exposed to Hyp (Fisher's exact test, n=6). FIG. 12E) STRING protein network analysis of 53 commonly upregulated genes (left) and 84 commonly downregulated genes (right) from Abx and GF mice relative to SPF controls, all fed KD and exposed to Hyp. (n=6). FIG. 12F) Volcano plots of pairwise comparisons labeling differentially expressed genes (log 2 fold change >2) in Abx (left) and GF (right) mice relative to SPF controls, all fed KD and exposed to Hyp. Differentially expressed genes shared by both Abx and GF conditions are labeled in red font. (n=6). Abx=treated with antibiotics (ampicillin, vancomycin, metronidazole, neomycin), GF=germ-free, SPF=specific pathogen-free (conventionally-colonized), KD=ketogenic diet, Hyp=intermittent hypoxia exposure.

FIG. 13A to FIG. 13I: Th1 cell Expansion Contributes to Bilophila-induced Impairments in Cognitive Behavior. FIG. 13A) Number of CD4+IFNy+CD3+Th1 cells in the lamina propria of GF, Bilophila wadsworthia-monocolonized (GF+Bilo) and Clostridium cocleatum monocolonized (GF+Clos) mice. (One-way ANOVA with Sidak, n=5-6). FIG. 13B) Percentages of IFNy+Th1 cells out of total CD3+ cell counts in the lamina propria of GF, GF+Bilo, and GF+Clos mice. (One-way ANOVA with Sidak, n=5-6). FIG. 13C) Representative flow cytometry plots of CD3+IFNy+Th1 cells and CD3+IL-17A+Th17 cells in the lamina propria of GF, GF+Bilo, and GF+Clos mice. FIG. 13D) Experimental timeline: GF+Bilo mice were injected intraperitoneally (with 0.5 mg for first bolus, followed by 0.25 mg for each subsequent bolus) anti-IL-12p40 or IgG2a isotype control every 2 days for 14 days and subjected to Barnes maze testing. FIG. 13E) Representative Barnes maze traces for GF+Bilo mice injected with anti-IL-12p40 or IgG2a isotype control. White lines indicate movement trajectories, whereas blue hues denote increasing durations of time spent at a particular location. Orange circles denote the escape hole. FIG. 13F) Per-mouse comparison of latency to enter the escape hole during the first and probe trial of the Barnes maze for GF+Bilo mice treated orally with anti-IL-12p40 or IgG2a isotype control. (Unpaired two-tailed Students t-test, n=15). FIG. 13G) Latency to enter the escape hole during the probe trial of the Barnes maze for GF+Bilo mice treated orally with anti-IL-12p40 or IgG2a isotype control. (Unpaired two-tailed Students t-test, n=15). FIG. 1311) Errors made during the probe trial of the Barnes maze for GF+Bilo mice treated orally with anti-IL-12p40 or IgG2a isotype control. (Unpaired two-tailed Students t-test, n=15). FIG. 131) Search strategy used during probe trial of the Barnes maze for GF+Bilo mice treated orally with anti-IL-12p40 or IgG2a isotype control. (n=14-15). Data are presented as mean±S.E.M. *p<0.05, **p<0.01. n.s.=not statistically significant.

FIG. 14A to FIG. 14D: Effects of anti-IL12p40 treatment on cognitive behavior and locomotion in the Barnes maze. FIG. 14A) Latency to enter the escape hole across six 300-second trials for GF+Bilo mice treated orally with anti-IL-12p40 or IgG2a isotype control. (Two-way ANOVA with Sidak, n=14-15). FIG. 14B) Errors made during as measured by number of incorrect nose pokes across six Barnes maze trials for GF+Bilo mice treated orally with anti-IL-12p40 or IgG2a isotype control. (Two-way ANOVA with Sidak, n=15). FIG. 14C) Average velocity of locomotion across trials in the Barnes maze for Bilo-monocolonized mice treated with anti-IL-12p40 or IgG2a isotype control. (Two-way ANOVA with Sidak, n=14-15). FIG. 14D) Total distance travelled across trials in the Barnes maze for Bilo-monocolonized mice treated with anti-IL-12p40 or IgG2a isotype control. (Two-way ANOVA with Sidak, n=14-15). Data are presented as mean±S.E.M. n.s.=not statistically significant.

FIG. 15A to FIG. 15L: There is no significant effect of acute intermittent hypoxia on cognitive behavior of female mice in the Barnes maze. FIG. 15A) Latency to enter (s) in response to mock and hypoxia (Hyp) treatment during control diet (CD) consumption (respectively SPF CD Mock and SPF CD Hyp)(n=10 SPF CD Mock mice; n=10 SPF CD Hyp mice). FIG. 15B) Distance in target quadrant (cm) for SPF CD Mock and SPF CD Hyp mice. FIG. 15C) Velocity (cm/s) for SPF CD Mock and SPF CD Hyp mice. FIG. 15D) Total distance (cm) for SPF CD Mock and SPF CD Hyp mice. FIG. 15E) Latency to enter (s) in response to mock and Hyp treatment during ketogenic diet (KD) consumption (respectively SPF KD Mock and SPF KD Hyp)(n=10 SPF KD Mock mice; n=10 SPF KD Hyp mice). FIG. 15F) Distance in target quadrant (cm) for SPF KD Mock and SPF KD Hyp mice. FIG. 15G) Velocity (cm/s) for SPF KD Mock and SPF KD Hyp mice. FIG. 15H) Total distance (cm) for SPF KD Mock and SPF KD Hyp mice. FIG. 15I) Latency to enter (s) in response to mock and Hyp treatment after antibiotic treatment (Abx) during ketogenic diet (KD) consumption (respectively Abx KD Mock and Abx KD Hyp)(n=10 Abx KD Mock mice; n=10 Abx KD Hyp mice). FIG. 15J) Distance in target quadrant (cm) for Abx KD Mock and Abx KD Hyp mice. FIG. 15K) Velocity (cm/s) for Abx KD Mock and Abx KD Hyp mice. FIG. 15L) Total distance (cm) for Abx KD Mock and Abx KD Hyp mice. Two-way ANOVA with Sidak's multiple comparison test (FIG. 15A to FIG. 15L), P<0.05, **P<0.01, ***P<0.001, ****P <0.0001. n.s.=not statistically significant. SPF=specific pathogen-free (conventionally-colonized), Abx (antibiotic treatment), CD=control diet, KD=ketogenic diet, normoxic treatment, Hyp=hypoxia treatment.

FIG. 16A to FIG. 16P: There is no significant effect of acute intermittent hypoxia on cognitive behavior of male mice in the novel object recognition task. FIG. 16A) Time in familiar object (sec) in response to mock and hypoxia (Hyp) treatment during control diet (CD) consumption (respectively SPF CD Mock and SPF CD Hyp)(n=6 SPF CD Mock mice; n=6 SPF CD Hyp mice). FIG. 16B) Time in novel object (sec) for SPF CD Mock and SPF CD Hyp mice. FIG. 16C) Entries in familiar object for SPF CD Mock and SPF CD Hyp mice. FIG. 16D) Entries in novel object for SPF CD Mock and SPF CD Hyp mice. FIG. 16E) Total distance for SPF CD Mock and SPF CD Hyp mice. FIG. 16F) Velocity (cm/s) for SPF CD Mock and SPF CD Hyp mice. FIG. 16G) Time in center (s) for SPF CD Mock and SPF CD Hyp mice. FIG. 1611) Entries in center SPF CD Mock and SPF CD Hyp mice. FIG. 161) Time in familiar object (sec) in response to mock and hypoxia (Hyp) treatment during ketogenic diet (KD) consumption (respectively SPF KD Mock and SPF KD Hyp)(n=6 SPF KD Mock mice; n=6 SPF KD Hyp mice). FIG. 16J) Time in novel object (sec) for SPF KD Mock and SPF KD Hyp mice. FIG. 16K) Entries in familiar object for SPF KD Mock and SPF KD Hyp mice. FIG. 16L) Entries in novel object for SPF KD Mock and SPF KD Hyp mice. FIG. 16M) Total distance for SPF KD Mock and SPF KD Hyp mice. FIG. 16N) Velocity (cm/s) for SPF KD Mock and SPF KD Hyp mice. FIG. 16O) Time in center (s) for SPF KD Mock and SPF KD Hyp mice. FIG. 16P) Entries in center SPF KD Mock and SPF KD Hyp mice. T-test (FIG. 16A to FIG. 16P), P<0.05, **P<0.01, ***P<0.001, ****P<0.0001. n.s.=not statistically significant. SPF=specific pathogen-free (conventionally-colonized), CD=control diet, KD=ketogenic diet, normoxic treatment, Hyp=hypoxia treatment.

FIG. 17A to FIG. 17P: There is no significant effect of acute intermittent hypoxia on cognitive behavior of male mice in the novel object location task. FIG. 17A) Time in familiar object (sec) in response to mock and hypoxia (Hyp) treatment during control diet (CD) consumption (respectively SPF CD Mock and SPF CD Hyp)(n=6 SPF CD Mock mice; n=6 SPF CD Hyp mice). FIG. 17B) Time in novel object (sec) for SPF CD Mock and SPF CD Hyp mice. FIG. 17C) Entries in familiar object for SPF CD Mock and SPF CD Hyp mice. FIG. 17D) Entries in novel object for SPF CD Mock and SPF CD Hyp mice. FIG. 17E) Total distance for SPF CD Mock and SPF CD Hyp mice. FIG. 17F) Velocity (cm/s) for SPF CD Mock and SPF CD Hyp mice. FIG. 17G) Time in center (s) for SPF CD Mock and SPF CD Hyp mice. FIG. 1711) Entries in center SPF CD Mock and SPF CD Hyp mice. FIG. 17I) Time in familiar object (sec) in response to mock and hypoxia (Hyp) treatment during ketogenic diet (KD) consumption (respectively SPF KD Mock and SPF KD Hyp)(n=6 SPF KD Mock mice; n=6 SPF KD Hyp mice). FIG. 17J) Time in novel object (sec) for SPF KD Mock and SPF KD Hyp mice. FIG. 17K) Entries in familiar object for SPF KD Mock and SPF KD Hyp mice. FIG. 17L) Entries in novel object for SPF KD Mock and SPF KD Hyp mice. FIG. 17M) Total distance for SPF KD Mock and SPF KD Hyp mice. FIG. 17N) Velocity (cm/s) for SPF KD Mock and SPF KD Hyp mice. FIG. 17O) Time in center (s) for SPF KD Mock and SPF KD Hyp mice. FIG. 17P) Entries in center SPF KD Mock and SPF KD Hyp mice. T-test (FIG. 17A to FIG. 17P), P<0.05, **P<0.01, ***P<0.001, ****P<0.0001. n.s.=not statistically significant. SPF=specific pathogen-free (conventionally-colonized), CD=control diet, KD=ketogenic diet, normoxic treatment, Hyp=hypoxia treatment.

### DETAILED DESCRIPTION OF THE INVENTION

In some aspects, the present disclosure provides methods of treating a subject afflicted with or at risk of developing cognitive impairment (e.g., hypoxia-induced cognitive impairment). Such methods include administration of an effective amount of a microbiome modulator to the subject. The microbiome modulator, in some embodiments, is a bacterial species, an antimicrobial agent, or a combination thereof (e.g., provided to the subject sequentially, provided to the subject simultaneously). The bacterial species can be Clostridium cocleatum, and the antimicrobial agent can be a beta-lactam antibiotic such as imipenem, cefoxitin, or ticarcillin.

In other aspects, the present disclosure also provides methods of selecting a subject having hypoxia-induced cognitive impairment and methods of assessing a degree (e.g., relative to a normal subject, relative to another time during the treatment, relative to pre-treatment) to which a subject is afflicted with hypoxia-induced cognitive impairment. These methods rely on determining a level of at least one biomarker associated with hypoxia-induced cognitive impairment. Such biomarkers include those that are expected to be higher in a subject not afflicted with hypoxia-induced cognitive impairment (e.g., Actb, Atg2a, Atp5d, Atp6v0e2, Camkv, Cldn11, Cldn5, Dctn4, Erbb3, Gabarap, Mag, Mapk11, Mbp, Micall1, Mobp, Nfasc, Nipa14, Pik3r2, Scn1b, Tubd1, Zfpm1) and those that are expected to be lower in a subject not afflicted with hypoxia-induced cognitive impairment (e.g., Adam7, Adcyap1, Adig, Adipoq, Adora2a, Adrb3, Aoc3, Avp, Baiap3, C3, Calb2, Car3, Cartpt, Cbin1, Cdo1, Ceacam10, Cidec, Cwc22, Defb20, Defb48, Dio2, Drd1, Ecel1, Etnppl, Fabp4, Fgf12, Fggy, Flvcr2, G0s2, Gad2, Glra1, Glra3, Gm42743, Gm44862, Gpx5, Hp, K1h11, Lcn8, Lgr5, Lyzf1, Marcks, mCG_18947, Meis1, Myo19, Pbx3, Penk, Plin1, Plin4, Pmch, Pnpla2, Prdm1, Retn, Retnla, Rgs9, Rrad, Rspo1, Scd1, Spink3, Spink1, Sslp1, Svs1, Svs4, Svs5, Svs6, Tacr1, Wisp1) as compared to a subject having hypoxia-induced cognitive impairment.

### Definitions

As used in the description, the words “a” and “an” can mean one or more than one. As used in the claims in conjunction with the word “comprising”, the words “a” and “an” can mean one or more than one. As used in the description, “another” can mean at least a second or more.

The term “treating” includes curing, relieving, or ameliorating to any extent a symptom of an illness or medical condition or preventing further worsening of such a symptom. For example, treating hypoxia-induced cognitive impairment includes making the cognitive impairment less severe.

The term “hypoxia-induced cognitive impairment” includes a lessening in the overall capacity or the speed of mental processes caused by less than adequate (e.g., inadequate, low, zero) oxygen supply to one or more tissues of a subject.

The term “microbiome modulator” includes members of the microbiome (e.g., one or more bacterial species that are or can be part of a subject's microbiome) as well as agents that cause changes in the microbiome (e.g., antibiotics, phages, bacterial species that are not normally part of a subject's microbiome).

The adjective “ketogenic-diet-suppressed” implies being negatively influenced by ketogenic diet. For example, a ketogenic-diet-suppressed bacterial species has a population that is decreased or eliminated when a subject shifts from a non-ketogenic diet to a ketogenic diet.

The adjective “ketogenic-diet-boosted” implies being positively influenced by ketogenic diet. For example, a ketogenic-diet-boosted bacterial species has a population that is increased when a subject shifts from a non-ketogenic diet to a ketogenic diet.

The term “level,” for example when forming a compound noun with a preceding word such as test, control, or reference, can denote a measurable value such as an amount, concentration, activity, maximum rate, Michaelis constant, half-maximal effective concentration, or half-maximal inhibitory concentration (e.g., of a biomarker or another tissue ingredient that is related to a biomarker). The term “level” also includes values such as presence or absence, which can be discrete when measured individually or can attain a more continuous character when measured collectively.

A “biomarker” can be anything that can be used as an indicator of a particular physiological state of an organism. For example, a biomarker can be a level of a metabolite, by-product, mRNA, peptide, polypeptide, or protein associated with a particular physiological state. When referring to a biomarker in this specification, no effective distinction is made between a peptide and a polypeptide. A “biomarker associated with hypoxia-induced cognitive impairment” is a biomarker that has a level in a subject having hypoxia-induced cognitive impairment that differs from its level in a subject (which can be the same subject before being afflicted with hypoxia-induced cognitive impairment) by more than a certain threshold (e.g., 5%, 10%, 15%, 20%, 25%, 30%, 35%, 40%, 45%, 50%, 55%, 60%, 65%, 70%, 75%, 80%, 85%, 90%, 95%, or 100% evaluated as a binary determination—ascertained with regard to only whether the difference falls onto one or the other side of the threshold without any regard to the quantitative extent of the difference). In some embodiments, this certain threshold is 20% evaluated as a binary determination. Any of these enumerated thresholds (e.g., 5% through 100%, and possibly also higher than 100%) can constitute a “predetermined threshold.”

The term “differs from” refers to a relative difference between two values. For example, if X differs by 10% relative to Y, then |X−Y|/|Y|=0.1, where the vertical bars (i.e., the pipes) denote the absolute values. Therefore, the term “differs from” encompasses both being “higher” (e.g., |110−100|/|100|=0.1; for X=110 and Y=100) and being “lower” (e.g., 190-1001/11001=0.1; for X=90 and Y=100). In some embodiments that are described with respect to a value differing from another by a certain amount, it might be sufficient to make a mere binary determination of whether the difference is on one side or the other side of a threshold value rather than any more fine-grained determination (e.g., for an embodiment in which X must differ by being higher by at least 20% relative to Y, it might be sufficient to determine whether X differs by some amount that is at least 20% instead of determining whether such a difference specifically is 60%, 90%, 120%, etc.). In embodiments that compare variables that are individually discrete or binary (e.g., presence, absence) rather than potentially continuous (e.g., amount, activity), the same “differs from” terminology can be used. In that case, if an individual measurement is compared, any non-zero difference in value indicates that the measurements are different (e.g., one is present, the other is absent). Again, in that case, if aggregate (rather than individual) measurements from a sample are compared, then the difference values indicate the population-level measurements (e.g., present at a level of 30 in the test sample vs. 60 in the reference sample (thereby differing by 50%), wherein the level can be the number of cells or any other measured level).

Depending on the compared values, the difference between the values can be referred to as a “test-reference differential” (e.g., when a test level differs from a reference level, which reference level can be either a level in a subject without a condition or a level in a subject before treatment) or as a “predetermined differential” (e.g., either equal to a predetermined threshold when comparing a test level to a level in a subject without a condition or equal to an acceptable incremental difference when comparing a test level after treatment of a subject to a level in the same subject before the treatment).

The phrase “pharmaceutically-acceptable carrier” as used herein means a pharmaceutically-acceptable material, composition or vehicle, such as a liquid or solid filler, diluent, excipient, solvent or encapsulating material. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “subject” refers to a mammal, including, but not limited to, a human or non-human mammal, such as a bovine, equine, canine, ovine, or feline.

Samples and Biomarker Detection

A biological sample can be obtained from an individual for use in the methods disclosed herein. The biological sample can be a biological fluid sample take from a subject. Examples of biological samples include urine, barbotage, blood, serum, plasma, tears, saliva, cerebrospinal fluid, tissue, lymph, synovial fluid, and sputum. A biological fluid sample can be whole blood, serum, or plasma. The sample can be diluted with a suitable diluent before the sample is analyzed. In some embodiments, the sample is a fecal sample obtained from the subject. The sample may be obtained from the subject using a variety of methods that are known in the art.

The methods disclosed herein can include detecting levels of a biomarker, in a subject or a biological sample obtained from the subject, and comparing them to their levels in a reference sample. Detecting alterations in the expression level of a biomarker can include measuring the level of protein or mRNA of the biomarker and comparing it to a control. Additionally, or alternatively, the methods can include genotyping or haplotyping the gene encoding the biomarker in a subject or a biological sample obtained from the subject, and comparing it with a control. In some embodiments, the biological sample is one that is isolated from the subject.

Compositions and Formulations

In some aspects, the invention relates to a composition (e.g., a food product or a pharmaceutical composition) comprising a microbiome modulator. The composition may comprise a pharmaceutically acceptable carrier. The composition may comprise probiotics. The pharmaceutical compositions disclosed herein may be delivered by any suitable route of administration, including orally, buccally, sublingually, parenterally, and rectally, as by powders, ointments, drops, liquids, gels, tablets, capsules, pills, or creams.

The compositions or formulations disclosed herein can include a carrier (e.g., a pharmaceutically acceptable carrier), nutrients, antimicrobial compounds, antifungal compounds, or antiviral compounds. Some of the antimicrobial compounds that can be used include capreomycins, including capreomycin IA, capreomycin IB, capreomycin IIA and capreomycin IIB; carbomycins, including carbomycin A; carumonam; cefaclor, cefadroxil, cefamandole, cefatrizine, cefazedone, cefazolin, cefbuperazone, cefcapene pivoxil, cefclidin, cefdinir, cefditoren, cefime, ceftamet, cefmenoxime, cefmetzole, cefminox, cefodizime, cefonicid, cefoperazone, ceforanide, cefotaxime, cefotetan, cefotiam, cefoxitin, cefpimizole, cefpiramide, cefpirome, cefprozil, cefroxadine, cefsulodin, ceftazidime, cefteram, ceftezole, ceftibuten, ceftiofur, ceftizoxime, ceftriaxone, cefuroxime, cefuzonam, cephalexin, cephalogycin, cephaloridine, cephalosporin C, cephalothin, cephapirin, cephamycins, such as cephamycin C, cephradine, chlortetracycline; chlarithromycin, clindamycin, clometocillin, clomocycline, cloxacillin, cyclacillin, danofloxacin, demeclocyclin, destomycin A, dicloxacillin, dirithromycin, doxycyclin, epicillin, erythromycin A, ethanbutol, fenbenicillin, flomoxef, florfenicol, floxacillin, flumequine, fortimicin A, fortimicin B, forfomycin, foraltadone, fusidic acid, gentamycin, glyconiazide, guamecycline, hetacillin, idarubicin, imipenem, isepamicin, josamycin, kanamycin, leumycins such as leumycin A1, lincomycin, lomefloxacin, loracarbef, lymecycline, meropenam, metampicillin, methacycline, methicillin, mezlocillin, micronomicin, midecamycins such as midecamycin A1, mikamycin, minocycline, mitomycins such as mitomycin C, moxalactam, mupirocin, nafcillin, netilicin, norcardians such as norcardian A, oleandomycin, oxytetracycline, panipenam, pazufloxacin, penamecillin, penicillins such as penicillin G, penicillin N and penicillin 0, penillic acid, pentylpenicillin, peplomycin, phenethicillin, pipacyclin, piperacilin, pirlimycin, pivampicillin, pivcefalexin, porfiromycin, propiallin, quinacillin, ribostamycin, rifabutin, rifamide, rifampin, rifamycin SV, rifapentine, rifaximin, ritipenem, rekitamycin, rolitetracycline, rosaramicin, roxithromycin, sancycline, sisomicin, sparfloxacin, spectinomycin, streptozocin, sulbenicillin, sultamicillin, talampicillin, teicoplanin, temocillin, tetracyclin, thostrepton, tiamulin, ticarcillin, tigemonam, tilmicosin, tobramycin, tropospectromycin, trovafloxacin, tylosin, and vancomycin, and analogs, derivatives, pharmaceutically acceptable salts, esters, prodrugs, and protected forms thereof.

The composition may be formulated for oral delivery. In some embodiments, the composition may comprise probiotics. In some embodiments, the compositions disclosed herein are food products. The composition may be in the form of a pill, tablet, or capsule. In some embodiments, the subject may be a mammal (e.g., a human). In some embodiments, the composition is self-administered. While it is preferred for a single composition to comprise all the bacteria to be administered, it will be recognized that for any of the various embodiments described herein, the combination of bacteria can similarly be administered in multiple compositions that together comprise the combination of bacteria. For example, the invention further provides kits comprising multiple compositions that together comprises bacteria of Clostridium genus (e.g., Clostridium cocleatum) as well as antimicrobial agents effective against bacteria of Bilophila genus (e.g., Bilophila wadsworthia).

In some embodiments, the composition is formulated for rectal delivery (e.g., a fecal sample). In some embodiments, the subject undergoes fecal microbiota transplant, wherein the transplant comprises a composition disclosed herein. Fecal microbiota transplantation (FMT), also commonly known as “fecal bacteriotherapy” represents a therapeutic protocol that allows the reconstitution of colon microbial communities. The process involves the transplantation of fecal bacteria from a healthy individual into a recipient. FMT restores colonic microflora by introducing healthy bacterial flora through infusion of a fecal sample, e.g., by enema, orogastric tube or by mouth in the form of a capsule containing freeze-dried material, obtained from a healthy donor. In some embodiments, the fecal sample is from a fecal bank.

In some aspects, the invention relates to a composition (e.g., a food product or a pharmaceutical composition) comprising Clostridium cocleatum bacteria and/or antibiotics against Bilophila wadsworthia. The composition may comprise a pharmaceutically acceptable carrier. The composition may comprise probiotics. The pharmaceutical compositions disclosed herein may be delivered by any suitable route of administration, including orally, bucally, sublingually, parenterally, and rectally, as by powders, ointments, drops, liquids, gels, tablets, capsules, pills, or creams. In certain embodiments, the pharmaceutical compositions are delivered generally (e.g., via oral administration). In certain other embodiments, the compositions disclosed herein are delivered rectally.

The composition may comprise any species of Clostridium, including, but not limited to, C. cocleatum. In some embodiments, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, of the bacteria in the composition are Clostridium bacteria

Compositions described herein may be used for oral administration to the gastrointestinal tract, directed at the objective of introducing the bacteria (e.g., the bacteria disclosed herein) to tissues of the gastrointestinal tract. The formulation for a composition (e.g., a probiotic composition) of the present invention may also include other probiotic agents or nutrients that promote spore germination and/or bacterial growth. An exemplary material is a bifidogenic oligosaccharide, which promotes the growth of beneficial probiotic bacteria. In some embodiments, the probiotic bacterial composition is administered with a therapeutically-effective dose of an (preferably, broad spectrum) antibiotic, or an anti-fungal agent. In some embodiments, the compositions described herein are encapsulated into an enterically-coated, delayed-release capsule or tablet. The enteric coating allows the capsule/tablet to remain intact (i.e., undissolved) as it passes through the gastrointestinal tract, until after a certain time and/or until it reaches a certain part of the GI tract (e.g., the small intestine). The delayed-release component prevents the “release” of the probiotic bacterial strain in the compositions described herein for a pre-determined period.

The composition may be a food product, such as, but not limited to, a dairy product. The dairy product may be cultured or a non-cultured (e.g., milk) dairy product. Non-limiting examples of cultured dairy products include yogurt, cottage cheese, sour cream, kefir, buttermilk, etc. Dairy products also often contain various specialty dairy ingredients, e.g. whey, non-fat dry milk, whey protein concentrate solids, etc. The dairy product may be processed in any way known in the art to achieve desirable qualities such as flavor, thickening power, nutrition, specific microorganisms and other properties such as mold growth control. The compositions of the present invention may also include known antioxidants, buffering agents, and other agents such as coloring agents, flavorings, vitamins, or minerals.

In some embodiments, the compositions of the present invention are combined with a carrier (e.g., a pharmaceutically acceptable carrier) which is physiologically compatible with the gastrointestinal tissue of the subject(s) to which it is administered. Carriers can be comprised of solid-based, dry materials for formulation into tablet, capsule or powdered form; or the carrier can be comprised of liquid or gel-based materials for formulations into liquid or gel forms. The specific type of carrier, as well as the final formulation depends, in part, upon the selected route(s) of administration. The therapeutic composition of the present invention may also include a variety of carriers and/or binders. In some embodiments, the carrier is micro-crystalline cellulose (MCC) added in an amount sufficient to complete the one gram dosage total weight. Carriers can be solid-based dry materials for formulations in tablet, capsule or powdered form, and can be liquid or gel-based materials for formulations in liquid or gel forms, which forms depend, in part, upon the routes of administration. Typical carriers for dry formulations include, but are not limited to trehalose, malto-dextrin, rice flour, microcrystalline cellulose (MCC) magnesium sterate, inositol, FOS, GOS, dextrose, sucrose, and like carriers. Suitable liquid or gel-based carriers include but are not limited to water and physiological salt solutions; urea; alcohols and derivatives (e.g., methanol, ethanol, propanol, butanol); glycols (e.g., ethylene glycol, propylene glycol, and the like). Preferably, water-based carriers possess a neutral pH value (i.e., pH 7.0). Other carriers or agents for administering the compositions described herein are known in the art, e.g., in U.S. Pat. No. 6,461,607.

In some embodiments, the composition further comprises other bacteria or microorganisms known to colonize the gastrointestinal tract. For example, the composition may comprise species belonging to the Firmicutes phylum, the Proteobacteria phylum, the Tenericutes phylum, the Actinobacteria phylum, or a combination thereof. Examples of additional bacteria and microorganisms that may be included in the subject compositions include, but are not limited to, Saccharomyces, Bacteroides, Eubacterium, Lactobacillus, Fusobacterium, Propionibacterium, Streptococcus, Enteroccus, Lactococcus and Staphylococcus, Peptostreptococcus. In certain embodiments, the composition is substantially free of bacteria that increase the risk of hypoxia-induced cognitive impairment. Such bacteria include Bilophila bacteria. Thus, in some embodiments, the composition is substantially free of Bilophila bacteria. A composition is substantially free of a bacterial type if that type makes up less than 10% of the bacteria in a composition, preferably less than 5%, even more preferably less than 1%, most preferably less than 0.5%, or even 0% of the bacteria in the composition.

In some embodiments, the composition comprises a fecal sample comprising at least one species of Clostridium. In some embodiments, the fecal sample is from a fecal bank. In some embodiments, the compositions may be added to a fecal sample prior to administration to the subject.

In some embodiments, provided herein are methods of treating or preventing a condition, such as seizures, by administering a composition (e.g., a fecal sample) that is enriched for at least one species of Clostridium to the subject. The fecal sample is enriched if at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1%, or at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, or at least 50% of the bacteria in the fecal sample is Clostridium. In some embodiments, the fecal sample is from a fecal bank. In some embodiments, the fecal sample is from a donor.

The composition may further comprise a nutrient. In some embodiments, the nutrient aids in the growth of bacteria (e.g., bacteria disclosed herein). In some embodiments, the nutrient is a lipid (e.g., lineoleic acid, stearic acid, or palmitic acid). In some embodiments, the nutrient may be conjointly administered with a composition disclosed herein. As used herein, the phrase “conjoint administration” refers to any form of administration of two or more different agents (e.g., a composition disclosed herein and a nutrient disclosed herein) such that the second agent is administered while the previously administered agent is still effective in the body. For example, the compositions disclosed herein and the nutrients disclosed herein can be administered either in the same formulation or in a separate formulation, either concomitantly or sequentially.

Actual dosage levels of the active ingredients in the pharmaceutical compositions may be varied to obtain an amount of the active ingredient that is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian can readily determine and prescribe the effective amount of the pharmaceutical composition required. For example, the physician or veterinarian could prescribe and/or administer doses of the compounds employed in the pharmaceutical composition at levels lower than that required in order to achieve the desired therapeutic effect and gradually increase the dosage until the desired effect is achieved.

Methods of Treatment and Methods of Use

In some aspects, the disclosure relates to methods of treating cognitive impairment (e.g., hypoxia-induced cognitive impairment). These methods include administration of an effective amount of a microbiome modulator to the subject. These methods also correspond to the use of a microbiome modulator in treatment of cognitive impairment.

The microbiome modulator can be administered as a pharmaceutical composition. For example, it can be in the form of an enteral formulation such as a tablet, capsule, paste, film, or solution, which may be modified to allow sustained release of the active ingredient.

In some embodiments, the microbiome modulator is a bacterial species (e.g., one that is part of the normal microbiome of a subject). The bacterial species can be a ketogenic-diet-suppressed bacterial species such as Clostridium cocleatum. In some embodiments, the microbiome modulator is an antimicrobial agent, such as an antibiotic effective against a ketogenic-diet-boosted bacterial species such as Bilophila wadsworthia. The antibiotic can be imipenem, clindamycin, metronidazole, cefoxitin, or ticarcillin.

Methods of Selecting a Subject

In some aspects, the disclosure relates to a method of selecting a subject that is afflicted with hypoxia-induced cognitive impairment. These methods include obtaining a level for a biomarker associated with hypoxia-induced cognitive impairment from a sample of a subject. If that level differs by more than a threshold from a control level for the same biomarker, then the subject is selected (e.g., as a subject that has hypoxia-induced cognitive impairment or as a subject that should receive a microbiome modulator as a therapy). The control level can be obtained from a sample taken from a healthy individual (e.g., a subject not having hypoxia-induced cognitive impairment) or it can be an already established value for a healthy individual.

The biomarker associated with hypoxia-induced cognitive impairment can be a biomarker that has a higher level (e.g., higher expression, higher mRNA level, higher protein level, higher peptide fragment level, higher enzyme activity) in a healthy individual as compared to a subject afflicted with hypoxia-induced cognitive impairment. Such biomarkers include Actb, Atg2a, Atp5d, Atp6v0e2, Camkv, Cldn11, Cldn5, Dctn4, Erbb3, Gabarap, Mag, Mapk11, Mbp, Micall1, Mobp, Nfasc, Nipa14, Pik3r2, Scn1b, Tubd1, and Zfpm1. The biomarker can also be one that has a lower level in a healthy individual as compared to a subject afflicted with hypoxia-induced cognitive impairment. Such biomarkers include Adam7, Adcyap1, Adig, Adipoq, Adora2a, Adrb3, Aoc3, Avp, Baiap3, C3, Calb2, Car3, Cartpt, Cbin1, Cdo1, Ceacam10, Cidec, Cwc22, Defb20, Defb48, Dio2, Drd1, Ecel1, Etnppl, Fabp4, Fgf12, Fggy, Flvcr2, G0s2, Gad2, Glra1, Glra3, Gm42743, Gm44862, Gpx5, Hp, Klhl1, Lcn8, Lgr5, Lyzf1, Marcks, mCG_18947, Meis1, Myo19, Pbx3, Penk, Plin1, Plin4, Pmch, Pnpla2, Prdm1, Retn, Retnla, Rgs9, Rrad, Rspo1, Scd1, Spink3, Spink1, Sslp1, Svs1, Svs4, Svs5, Svs6, Tacr1, and Wisp1. For each of these two types of biomarkers, also included are their corresponding RNA forms and peptide/polypeptide/protein forms, as well as any other metabolites that have levels correlating with such forms (e.g., because of being a reactant or a product for a protein that acts as an enzyme, or because of being part of the same biochemical pathway as an RNA or protein).

Methods of Treating a Selected Subject

In some aspects, the disclosure relates to a method of treating a subject afflicted with hypoxia-induced cognitive impairment by selecting a subject that has hypoxia-induced cognitive impairment and then administering an effective amount of a microbiome modulator to the subject. The subject having hypoxia-induced cognitive impairment can be selected based on the subject having a level for a biomarker associated with hypoxia-induced cognitive impairment that differs by more than a threshold from a control level for the same biomarker. In some such treatment methods, a sample from a test subject can be actively tested to determine the level of the biomarker, while in some alternative methods, the level of the biomarker can already be known because of a prior determination.

Methods of Obtaining a Prognostic Indicator for a Treated Subject

In some aspects, the disclosure relates to a method of obtaining a prognostic indicator of hypoxia-induced cognitive impairment in a subject that receives a dosage of a microbiome modulator. In these methods, the subject is administered a test dose of the microbiome modulator, after which a level for a biomarker associated with hypoxia-induced cognitive impairment is obtained from a sample of the subject. Once this level is obtained, it can be compared to a reference level to determine whether it differs from the reference level by more than a certain differential.

In one variant of these methods, the reference level is representative of the level of the biomarker in a healthy individual that does not have hypoxia-induced cognitive impairment. Therefore, the difference between the tested level and the reference level in these methods can be used to assess how close to being healthy the treated subject is as a treatment regimen is continued.

In another variant of these methods, the reference level is obtained from the same subject before administration of the test dose of the microbiome modulator. In such methods, the difference between the test level and the reference level can be compared to an acceptable incremental threshold (e.g., 5%, 10%, 15%) to determine whether the treatment regimen results in a desirable improvement of the hypoxia-induced cognitive impairment.

In any of the comparisons between a test value and a threshold value, in some embodiments it is sufficient to determine whether the difference is meets the threshold (e.g., is equal to or higher than the threshold) or does not meet it.

In any of these methods (e.g., treatment, selection, prognosis), the subject can be a human (e.g., a male human). In some embodiments, the subject is initially tested for dihydrotestosterone levels, and is treated once it is determined that the dihydrotestosterone levels of the subject are at a level that exacerbates hypoxia-induced cognitive impairment.

### EXAMPLES

### Example 1: The Gut Microbiota Modulates Environmental Risk for Cognitive Impairment Results

The Ketogenic Diet Exacerbates Hypoxia-Induced Cognitive Impairment

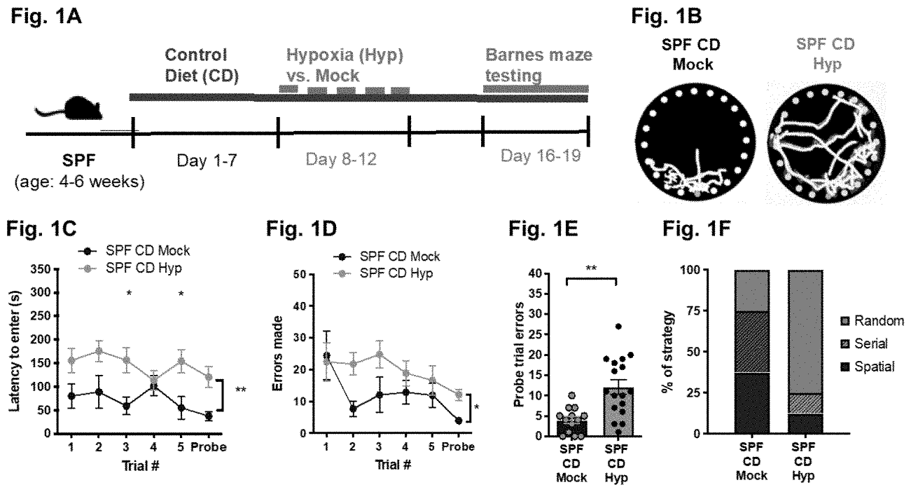

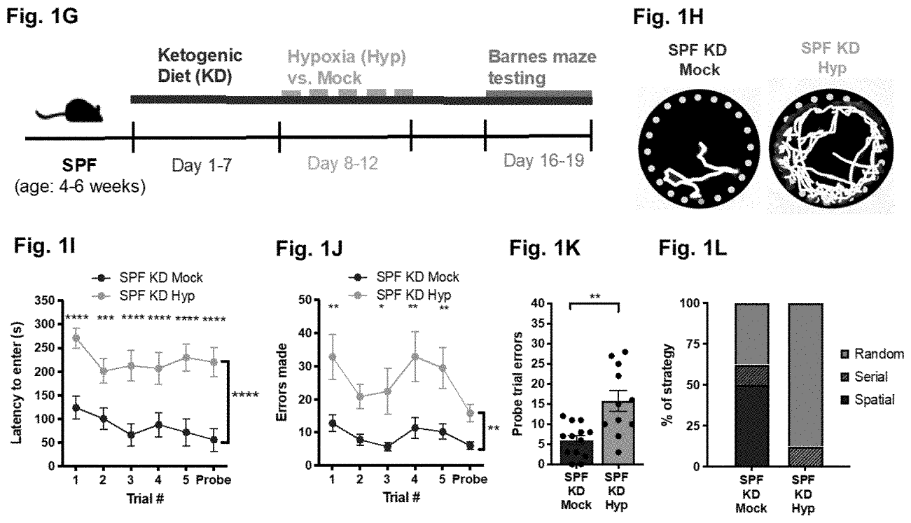

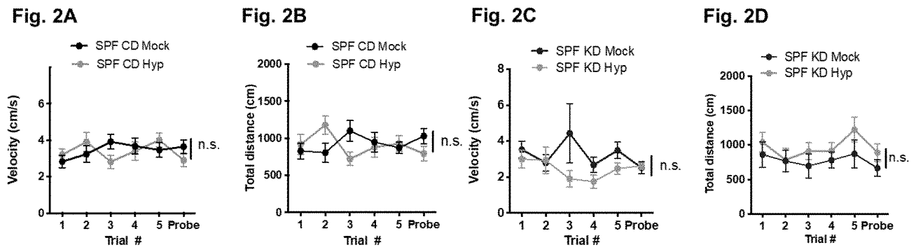

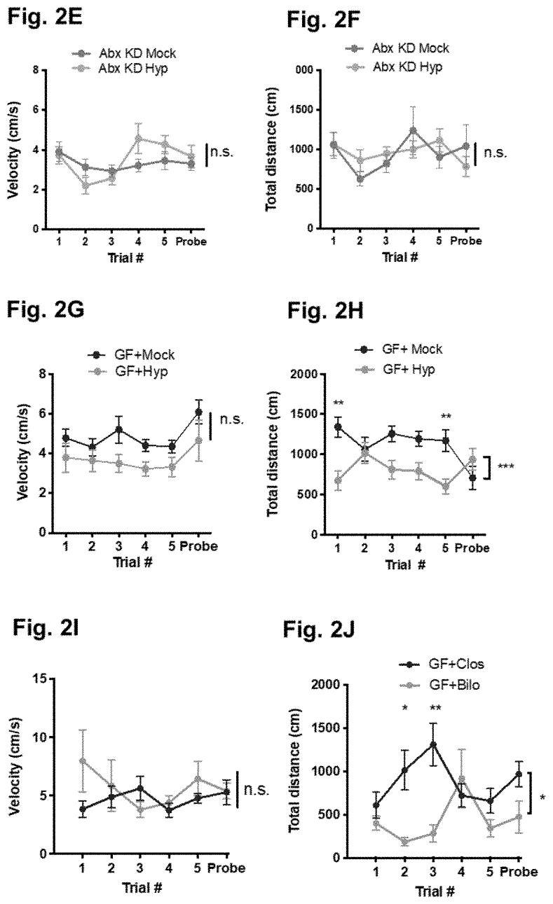

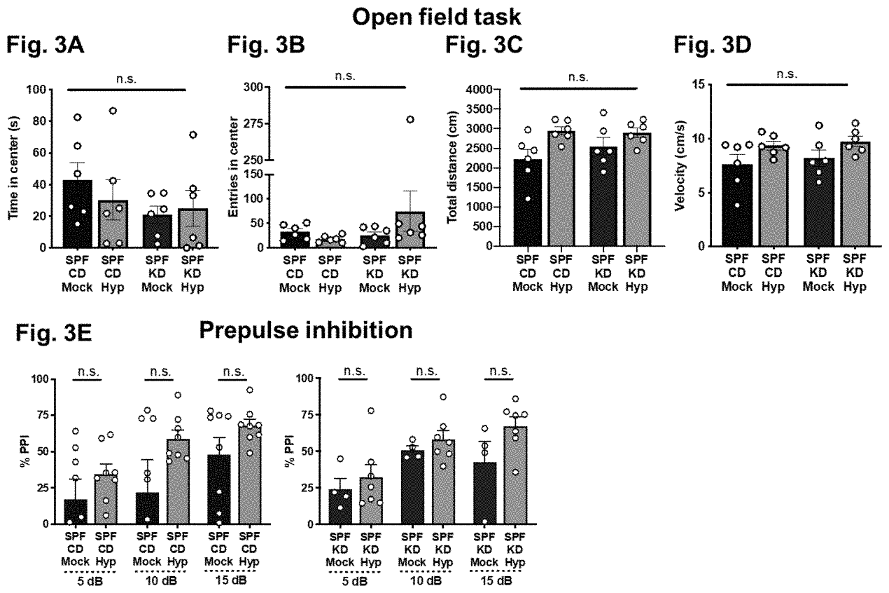

Hypoxia is an environmental risk factor for cognitive impairment associated with high altitude exposure, sleep apnea, vascular dementia, and Alzheimer's disease, among many other pathological conditions. In order to determine how acute intermittent hypoxia impacts cognitive behavior, conventional mice (specific pathogen-free, SPF) were subjected to 6 hours of restricted 12% oxygen (normobaric hypoxia, Hyp) or ambient 21% oxygen (normoxia, Mock) daily for five days. They were then given a 4 day recovery period, followed by behavioral testing for spatial learning and memory in the Barnes maze task (FIG. 1A). Consistent with prior literature, mice exposed to Hyp exhibited impaired cognitive behavior in the Barnes maze, as indicated by increases in latency to enter the escape box, errors made, and use of random search strategy as compared to Mock controls (FIG. 1B to FIG. 1F). These impairments were observed even on the first trial of testing, suggesting cognitive impairment that can include disrupted learning and memory, in additional to other underlying cognitive processes. There were no significant differences in velocity or total distance travelled in the Barnes maze (FIG. 2A and FIG. 2B), suggesting no confounding abnormalities in motor function. There were also no differences in performance in the open field test and pre-pulse inhibition task, suggesting no alterations in anxiety-related exploration or sensorimotor gating (FIG. 3A to FIG. 3E). These findings indicate that acute intermittent hypoxia impairs cognitive behavior in the Barnes maze.

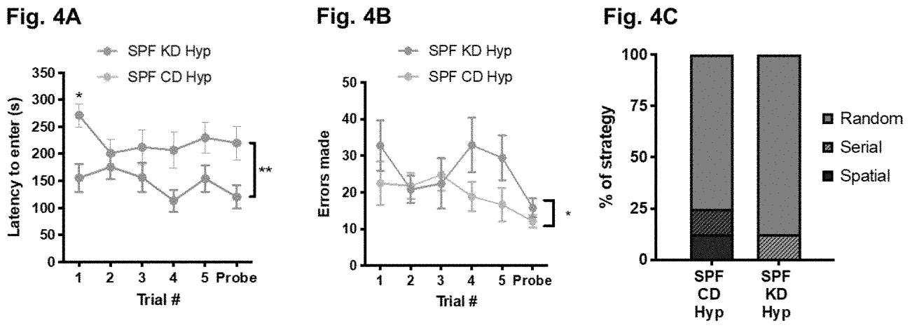

The high-fat, high-sugar “Western” diet disrupts cognitive ability in humans and rodents, particularly in response to physical or psychosocial stress. To determine whether the high-fat, low-carbohydrate ketogenic diet (KD) modifies cognitive behavior, SPF mice were pre-treated with the KD and subjected to Mock or Hyp as described above (FIG. 1G). Mice fed the KD exhibited no significant difference in cognitive behavior in the Barnes maze, as compared to mice fed the vitamin and mineral-matched control diet (CD) (FIG. 1A to FIG. 1L, SPF CD Mock vs. SPF KD Mock), indicating that the KD alone has no overt effect on cognitive behavior in the Barnes maze. Notably, however, mice fed the KD and exposed to Hyp (SPF KD Hyp) exhibited a substantial increase in latency to enter the escape box, errors made, and random search strategy as compared to Mock controls fed the KD (SPF KD Mock) (FIG. 111 to FIG. 1L). The Hyp-induced behavioral impairment was significantly more severe in KD-fed mice than in CD-fed mice (FIG. 1A to FIG. 1L, and FIG. 4A to FIG. 4C). There was no significant difference across experimental groups in velocity or total distance traveled in the Barnes maze (FIG. 2C and FIG. 2D), or in performance in the open field and prepulse inhibition tasks (FIG. 3A to FIG. 3E). These results indicate that the KD potentiates the adverse effects of Hyp on cognitive behavior, and further highlight synergistic interactions between diet and hypoxic stress as environmental risk factors for cognitive impairment.

Ketogenic Diet- and Hypoxia Associated Alterations in the Gut Microbiota Impair Cognitive Behavior

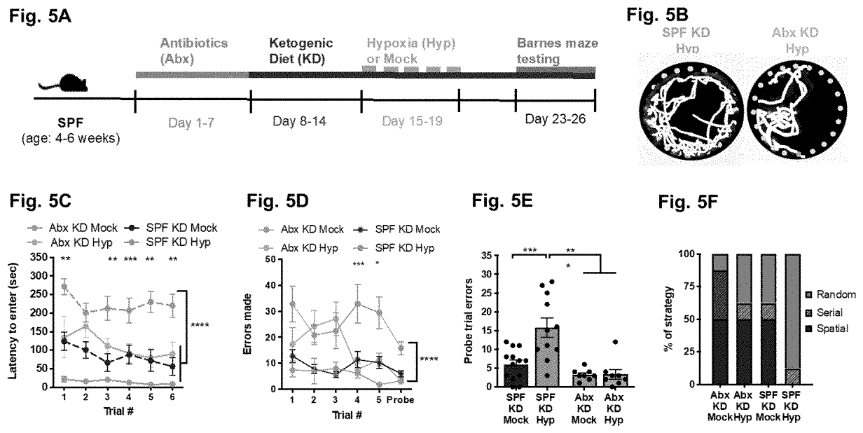

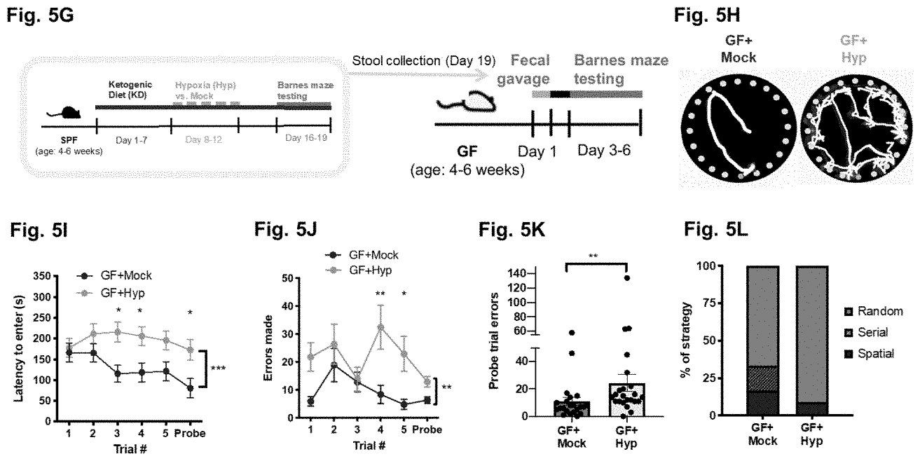

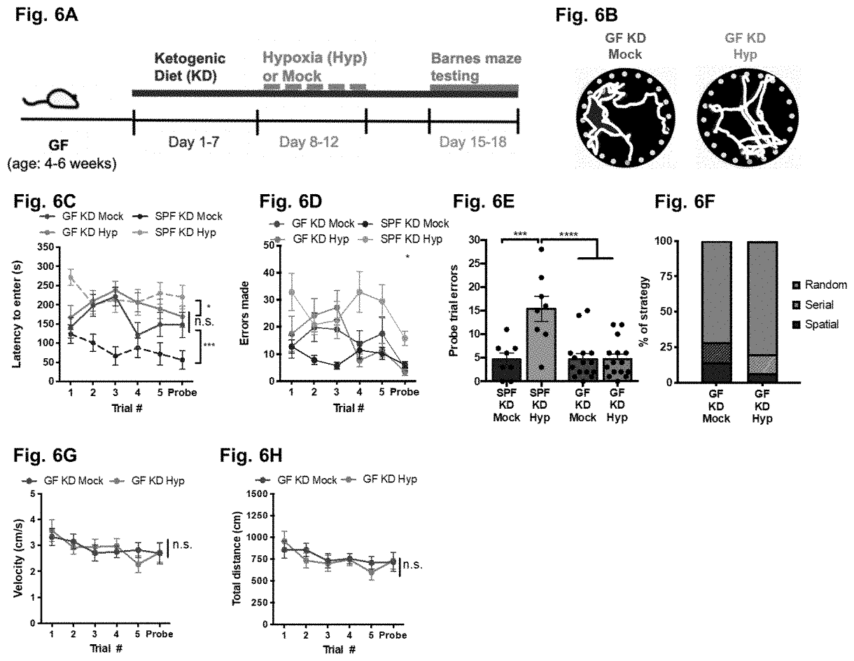

Environmental factors, including diet and stress, play important roles in shaping the composition and function of the gut microbiota. To determine whether the gut microbiota contributes to KD and Hyp-induced disruptions in cognitive behavior, SPF mice were pre-treated with broad-spectrum antibiotics (Abx) to deplete the gut microbiota prior to KD and Hyp exposure (FIG. 5A). Compared to vehicle-treated controls, KD- and Hyp-exposed mice that were pre-treated with Abx exhibited improved cognitive performance, as indicated by decreases in the latency to enter the escape box, errors made, and use of random search strategy (FIG. 5B to FIG. 5F, SPF KD Hyp vs. Abx KD Hyp). Results for each of these behavioral parameters were comparable to those seen in Mock controls fed the KD (FIG. 5B to FIG. 5F, FIG. 2E, and FIG. 2F; Abx KD Hyp vs. SPF KD Mock), suggesting that depletion of the microbiota abrogated KD and Hyp-induced impairments in cognitive behavior. Consistent with this, germ-free (GF) mice fed the KD were resistant to Hyp-induced cognitive impairments in the Barnes maze (FIG. 6A to FIG. 611). Compared to KD-fed SPF mice, however, GF mice fed the KD exhibited worse cognitive behavior at baseline, suggesting detrimental effects of KD particularly in mice raised GF. These data suggest that depletion of the gut microbiota prevents the synergistic effects of KD and Hyp on cognitive impairment.

To further test whether microbiota alterations in response to KD and Hyp contribute to disruptions in cognitive behavior, GF mice fed standard chow were transplanted with fecal microbiota from donor SPF mice fed KD and exposed to either Hyp or Mock (GF+Hyp and GF+Mock, respectively) (FIG. 5G). Compared to controls colonized with the KD and Mock-associated microbiota, mice colonized with KD and Hyp-associated microbiota exhibited poor cognitive performance (FIG. 511 to FIG. 5L, FIG. 2G, and FIG. 211), akin to that seen in mice that were actually fed KD and exposed to Hyp (FIG. 111 to FIG. 1L). Relative to SPF mice, transplanted control mice exhibited impaired performance on the first trial that improved with subsequent trials of the task, which may reflect initial confounding effects of GF status on behavior. Taken together, these results indicate that i) the KD potentiates Hyp-induced impairments in cognitive behavior (FIG. 1A to FIG. 1L, and FIG. 4A to FIG. 4C), ii) depletion of the microbiota prevents the adverse synergistic effects of KD and Hyp on cognitive behavior (FIG. 5A to FIG. 5F), and iii) transplantation of the KD- and Hyp-associated microbiota into naïve mice impairs cognitive behavior (FIG. 5G to FIG. 5L). These results strongly suggest that changes in the gut microbiota contribute to the KD- and Hyp-induced impairments in cognitive behavior.

Bilophila is Enriched by the Ketogenic Diet and Hypoxia and Impairs Cognitive Behavior

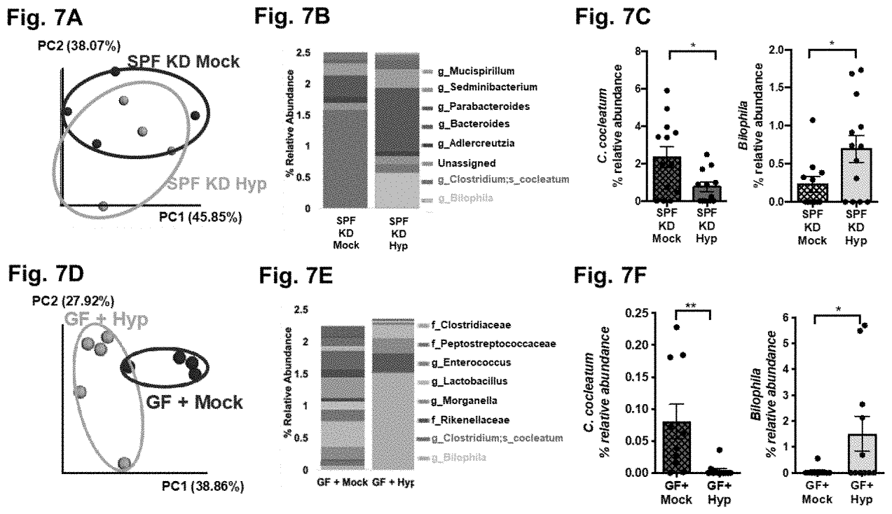

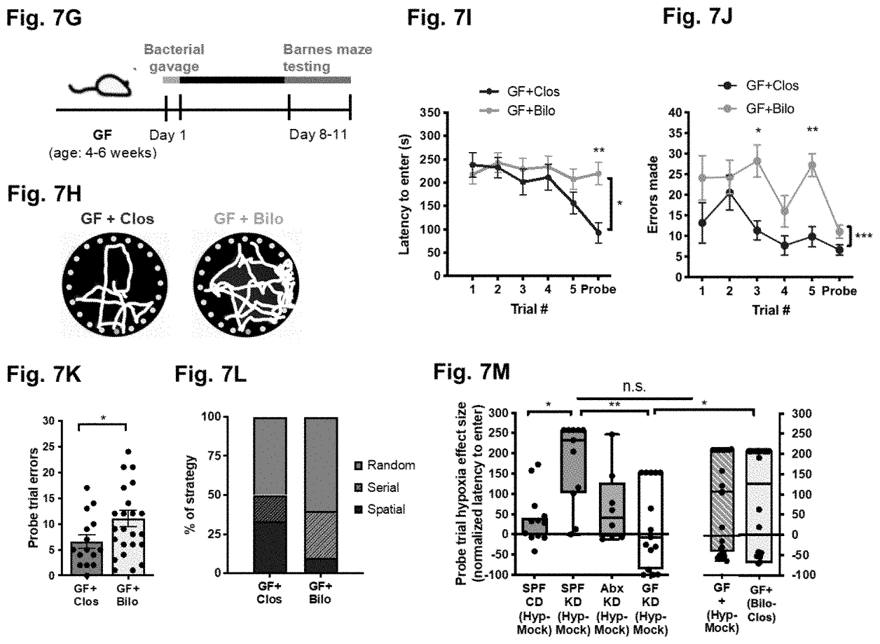

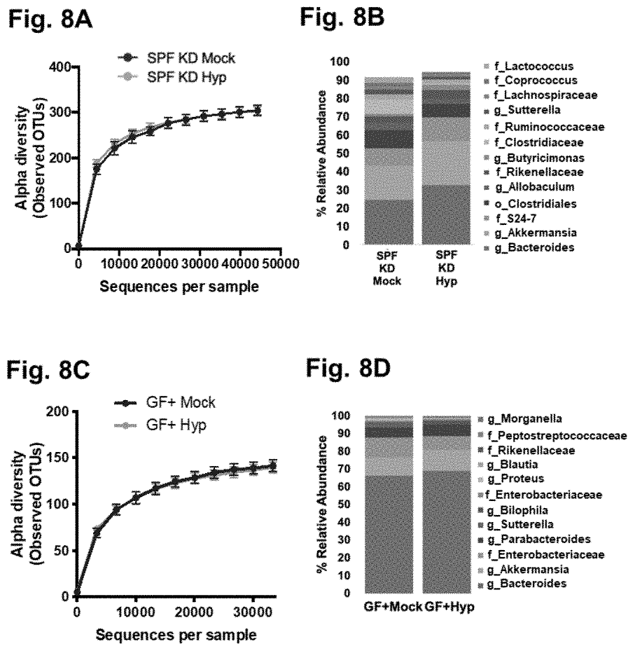

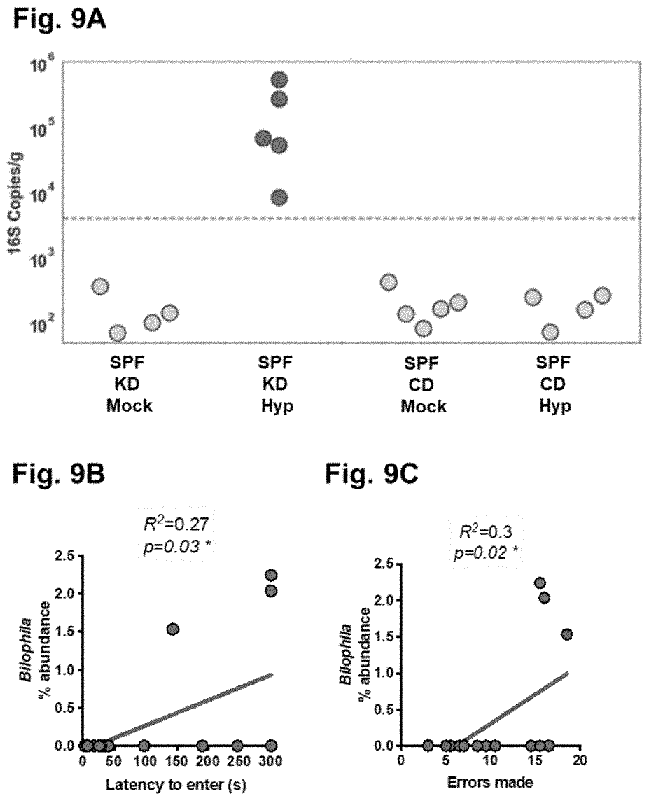

To identify candidate microbial taxa that may be responsible for promoting KD- and Hyp-induced abnormalities in cognitive behavior, fecal microbiota were sequenced from SPF mice fed KD and exposed to Hyp or Mock (FIG. 111 to FIG. 1L), as well as from the mice transplanted with the corresponding microbiota from those SPF mice (FIG. 5G to FIG. 5L). While there were no global alterations in the microbiota of mice fed KD and exposed to Hyp (FIG. 7A, and FIG. 8A to FIG. 8D), select bacterial taxa were significantly altered in the Hyp group, compared to Mock controls (FIG. 7B and FIG. 7C). In particular, the relative abundances of Clostridium cocleatum were reduced in Hyp-exposed animals fed KD, while Bilophila species were elevated in Hyp-exposed animals fed KD (FIG. 7B and FIG. 7C). Quantification of 16S rRNA gene counts revealed that Bilophila species were enriched by absolute abundance, particularly in mice exposed to both KD and Hyp, but not in mice exposed to either KD or Hyp alone (FIG. 9A). This aligns with the observed synergistic effects of KD and Hyp on cognitive impairment (FIG. 1A to FIG. 1L, and FIG. 4A to FIG. 4C), and with previous reports that dietary fat favors the growth of Bilophila, which is an obligate anaerobe. Similar reductions in C. cocleatum and increases in Bilophila were seen in mice transplanted with KD and Hyp-associated microbiota (FIG. 7D to FIG. 7F), where Bilophila levels exhibited modest, but statistically significant, correlations with the severity of cognitive impairment in the Barnes maze (FIG. 9B and FIG. 9C). Overall, these results reveal that KD and Hyp together enrich Bilophila and impair cognitive behavior.

To test whether Bilophila may contribute to the cognitive impairment seen in response to KD and Hyp, GF mice fed standard chow were monocolonized with Bilophila wadsworthia and tested for cognitive behavior, relative to controls colonized with C. cocleatum (FIG. 7G). B. wadsworthia was selected because it exhibited the highest sequence identity to the Bilophila operational taxonomic units elevated in mice fed KD and exposed to Hyp (FIG. 7B and FIG. 7C). Mice colonized with B. wadsworthia exhibited impaired cognitive behavior compared to GF and C. cocleatum-colonized controls, with no overt deficits in motor ability (FIG. 7I to FIG. 7L, FIG. 2I, and FIG. 2J). The effect of B. wadsworthia on cognitive impairment during the probe trial of the Barnes maze assay was comparable to that seen with exposure to KD and Hyp and with transplantation of the KD- and Hyp-associated microbiota (FIG. 7M). These data reveal that monocolonization with B. wadsworthia phenocopies the adverse effects of the KD- and Hyp-associated microbiota on cognitive behavior.

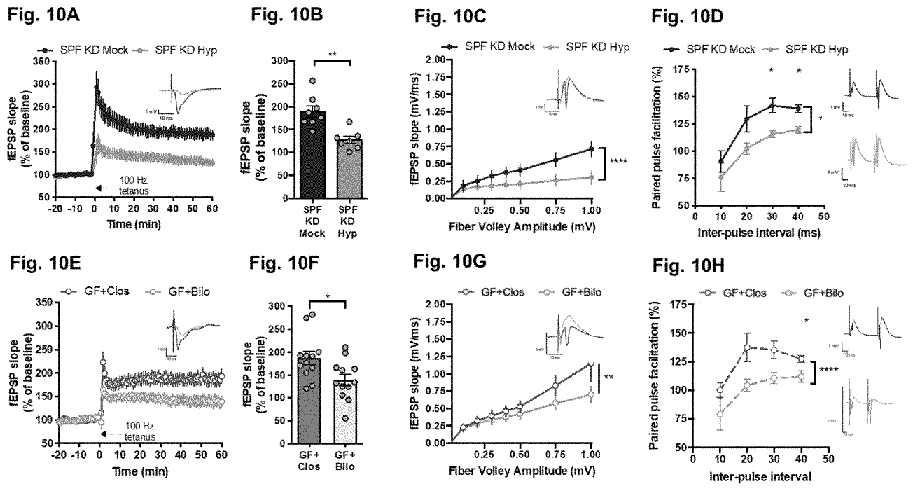

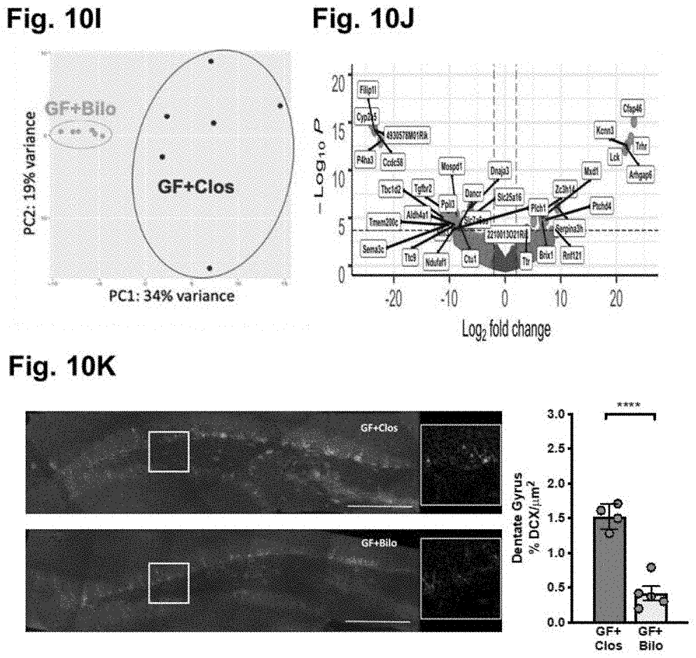

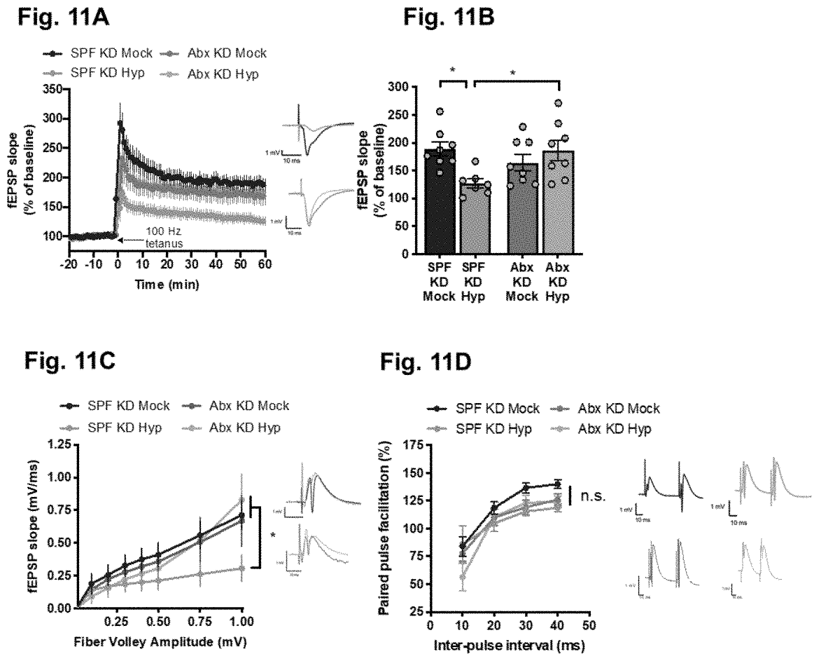

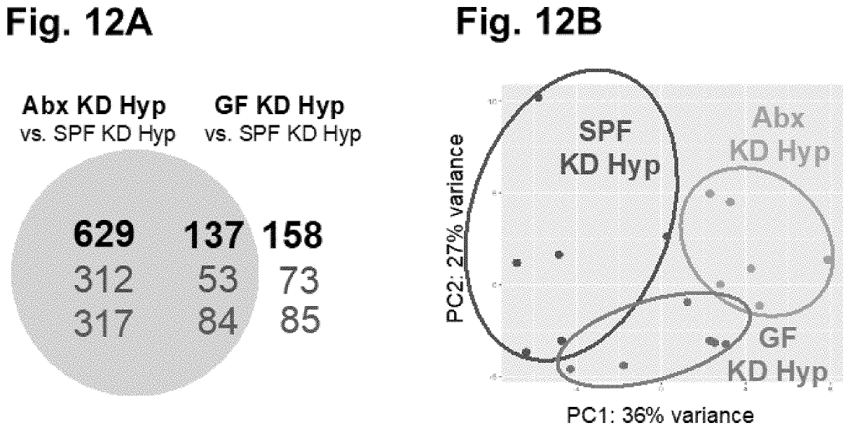

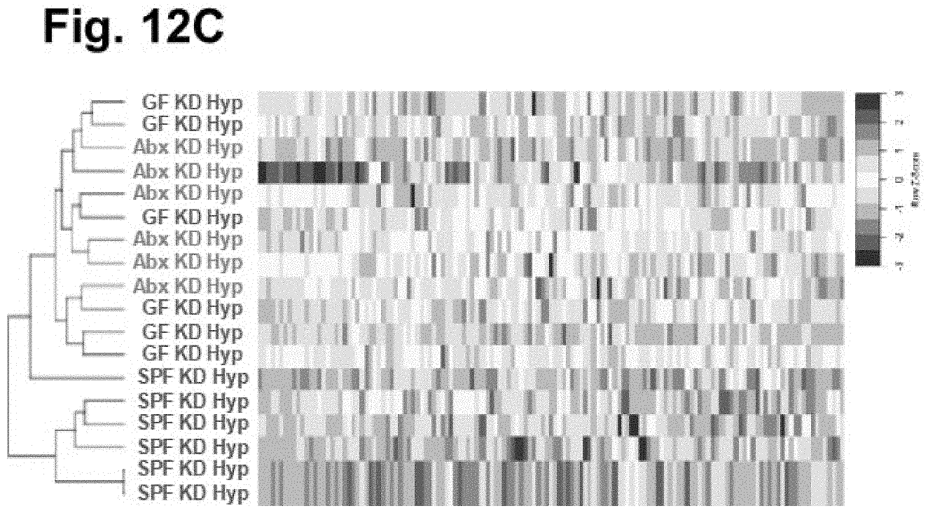

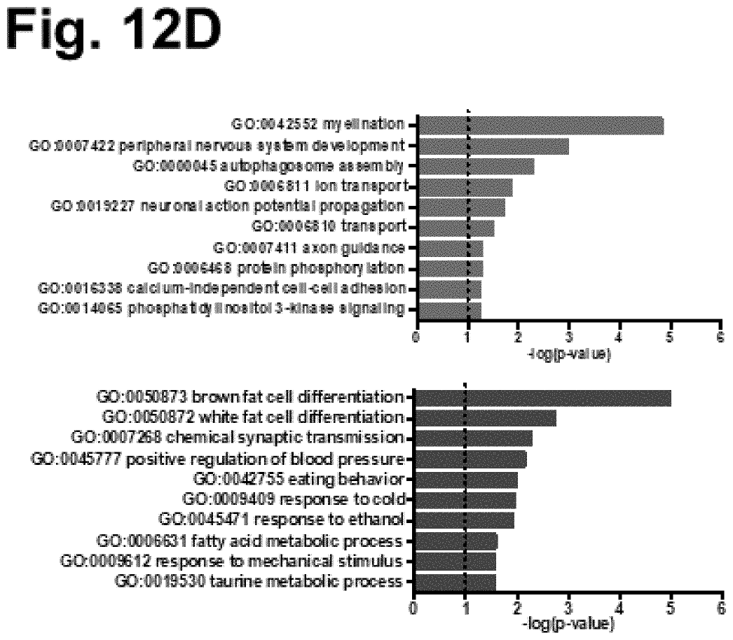

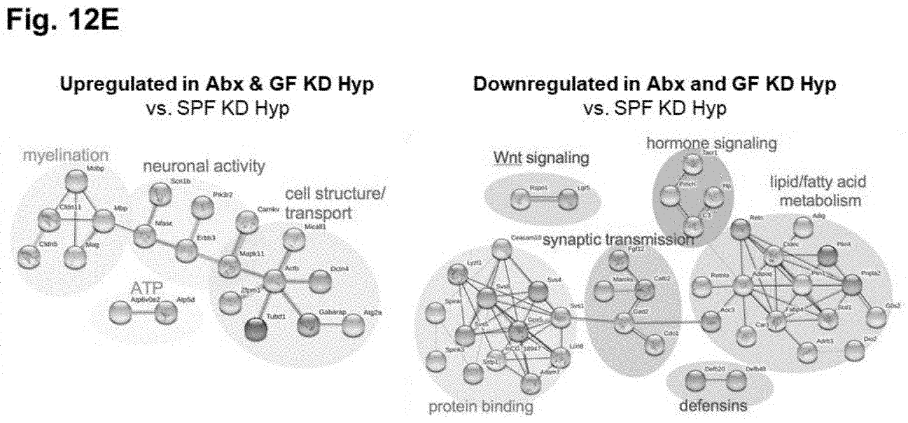

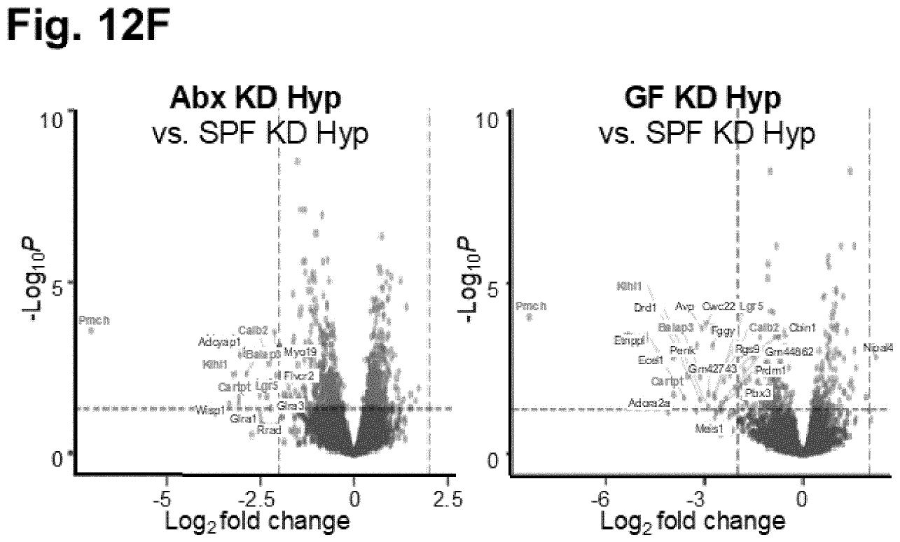

The Gut Microbiota, and Bilophila in Particular, Modulates Hippocampal Activity The hippocampus is sensitive to alterations in diet and hypoxic stress, and is a critical site for learning and memory. To study effects of the KD and Hyp, and potential roles for the microbiota, on hippocampal physiology, field potential recordings were acquired from acute hippocampal slices from SPF mice fed KD and exposed to Hyp or Mock, as well as from mice colonized with B. wadsworthia or C. cocleatum. Compared to Mock-exposed controls, KD-fed mice that were exposed to Hyp exhibited significant reductions in hippocampal long-term potentiation (LTP, FIG. 10A and FIG. 10B), fiber volley amplitude relative to field excitatory postsynaptic potential (fEPSP) slope, as a measure of basal synaptic strength (FIG. 10C), and paired-pulse facilitation (FIG. 10D). Pre-treatment with Abx diminished these abnormalities in hippocampal activity (FIG. 11A to FIG. 11D), suggesting that the gut microbiota contributes to the adverse effects of KD and Hyp on hippocampal physiology. Consistent with this, mice monocolonized with B. wadsworthia exhibited reduced hippocampal LTP (FIG. 10E and FIG. 10F), reduced fiber volley amplitude vs. fEPSP slope (FIG. 10G), and reduced paired-pulse facilitation (FIG. 1011) when compared to C. cocleatum-colonized controls. The alterations in hippocampal activity induced by B. wadsworthia colonization were comparable to those seen in mice exposed to KD and Hyp, suggesting that B. wadsworthia contributes to the adverse effects of KD and Hyp on hippocampal function. The microbiota-dependent alterations in hippocampal activity were further associated with microbiota-dependent changes in hippocampal gene expression (FIG. 12A to FIG. 12F). In particular, colonization with B. wadsworthia resulted in widespread transcriptomic alterations in the hippocampus relative to those seen in C. cocleatum-colonized controls (FIG. 10I). Particular genes that were differentially expressed in response to B. wadsworthia colonization included subsets related to neuronal excitation (CBLN1, CC2D 1A, GRIK4, SYT2, SYT9), ubiquitination (BAP1, COPSE, FBXO4, FBXO42, NDUFAF 5, USP53), and the immune response (HMGB1, IL16, NFKBID, TRAFD1, SPG21) (FIG. 10J). Moreover, immunofluorescence staining for doublecortin (DCX), a marker for hippocampal neurogenesis, revealed reduced density of DCX-positive cells in the dentate gyrus of mice colonized with B. wadsworthia compared with C. cocleatum-colonized controls (FIG. 10K), suggesting impaired neurogenesis. Overall, these results indicate that colonization with B. wadsworthia alters hippocampal physiology, and further suggest that microbiota-dependent impairments in cognitive behavior may be due, at least in part, to microbiota-dependent disruptions in hippocampal function.

Th1 Cell Expansion Contributes to Bilophila-Induced Impairments in Cognitive Behavior

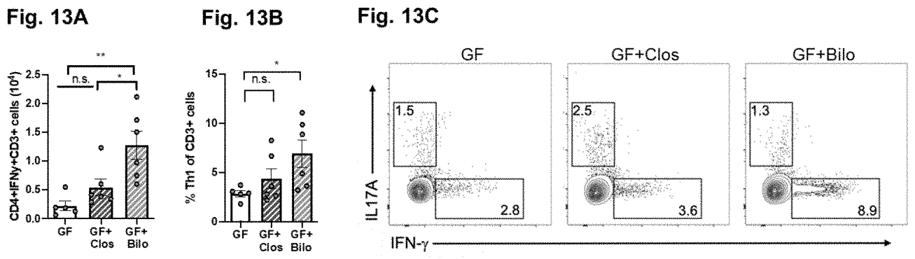

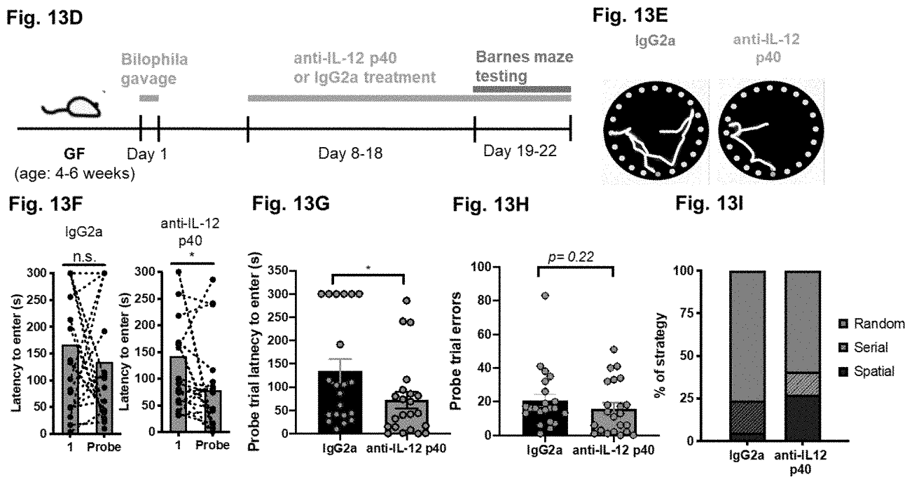

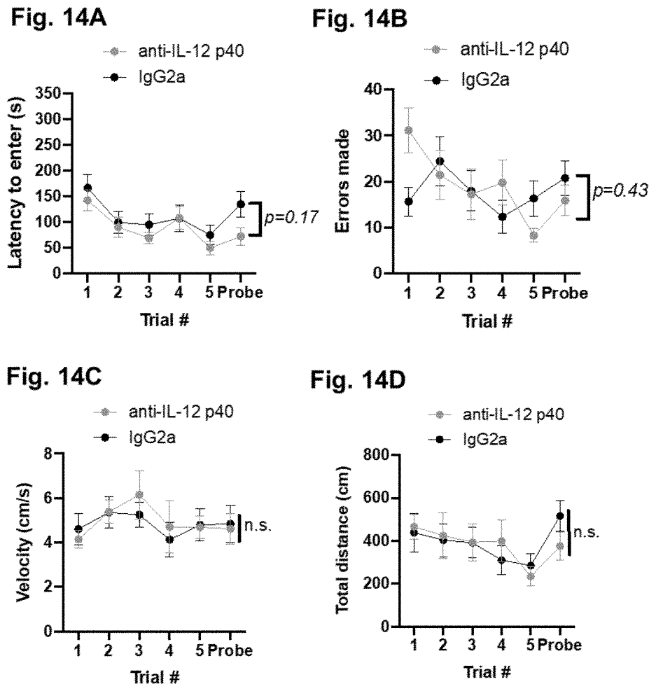

B. wadsworthia promotes the expansion of pro-inflammatory T helper type I (Th1) cells via increases in the cytokine IL-12p40, and IFNγ production by Th1 cells is associated with impairments in cognitive behavior. To determine if Th1 cell induction by B. wadsworthia mediates its adverse effects on cognitive behavior, B. wadsworthia-colonized mice were pre-treated systemically with anti-IL-12p40 neutralizing antibody to inhibit Th1 expansion and then tested for cognitive behavior in the Barnes maze. Indeed, B. wadsworthia-colonized mice exhibited increased numbers of CD4+IFNγ+CD3+Th1 cells in the colonic lamina propria relative to GF and C. cocleatum-colonized controls (FIG. 13A and FIG. 13B), and pre-treatment with anti-IL-12p40, but not IgG2a, prevented the increases in Th1 cells seen in B. wadsworthia-colonized mice. Notably, compared to IgG2a-treated controls, mice treated with anti-IL-12p40 antibody exhibited improved cognitive behavior, as denoted by reductions in latency to enter the escape box, errors made and use of random search strategy, with no differences in total distance traveled or velocity. These results suggest that B. wadsworthia promotes cognitive impairment via the expansion of pro-inflammatory Th1 cells. To further determine if Th1 cells mediate the adverse effects of KD and Hyp on cognitive behavior, mice fed the KD were pre-treated anti-IL-12p40 or IgG2a and then exposed to Hyp or Mock. KD-fed mice exposed to Hyp exhibited elevated intestinal Th1 cells compared to Mock controls, and anti-IL-12p40 treatment prevented the KD and Hyp-induced increases in Th1 cells. Moreover, KD- and Hyp-exposed mice that were treated with anti-IL-12p40 exhibited improved cognitive behavior, with decreases in latency to enter the escape box, errors made and use of random search strategy, relative to IgG2a-treated controls. Altogether, these results reveal that B. wadsworthia impairs cognitive behavior by increasing Th1 cells, and further suggest that microbiota-dependent increases in Th1 cells mediate the adverse effects of KD and Hyp on cognitive behavior.

### Discussion

Proof-of-concept studies have reported that the complete absence of the microbiota results in widespread alterations in animal behavior, including learning and memory, but whether microbiota is altered by physiologically-relevant risk factors for cognitive dysfunction and whether there are select microbial species that are causally linked to cognitive impairment was poorly understood. Results from this study reveal that the gut microbiota is altered by synergistic interactions between environmental risk factors for cognitive impairment. They further demonstrate that select diet- and stress-responsive bacteria from the gut microbiota disrupt hippocampal function and cognitive behavior, likely via the induction of pro-inflammatory immune cells.

In particular, we find that the high-fat, low carbohydrate KD exacerbates the detrimental effects of acute intermittent Hyp on cognitive behavior. This aligns with the so-called “two-hit” or “multiple-hit” hypotheses for neurological and neurodegenerative diseases, wherein multiple genetic and/or environmental risk factors interact to accelerate or predispose to symptoms of disease, including age-related cognitive decline. Indeed, both high-fat diet and hypoxia are associated with cognitive impairment across studies of humans and animal models. However, the ketogenic diet and select ketone bodies, such as beta-hydroxybutyrate, have been recently explored as potential treatments for behavioral symptoms of Alzheimer's disease. Results from this study suggest that when combined with other environmental stressors, the KD can be detrimental to hippocampal function and cognitive behavior. Further research is warranted to uncover the molecular bases for interactions between varied genetic and environmental risk factors for cognitive impairment.

Data from this study indicate that alterations in the gut microbiota contribute to the adverse effects of the KD and Hyp on cognitive behavior in the Barnes maze task. One important consideration is the biological bases of the abnormalities observed in the behavioral task. Adverse effects of the KD and Hyp are seen even during the first trial of the behavioral test that persist through the sixth and last trial. While we observe no overt differences in anxiety-related exploration in the open field, acoustic startle response, and sensorimotor gating in the prepulse inhibition task, the early abnormalities in the behavioral task suggest that the KD and Hyp together induce cognitive issues that extend beyond disruptions in learning and working memory. Depletion of the microbiota diminishes the behavioral abnormalities, even on the first trial of testing, and also improves hippocampal LTP, suggesting effects on hippocampal-dependent learning and memory as well other aspects of cognitive performance. The gain of function studies using the microbiota transplant and bacterial monocolonization paradigms reveal that the colonized control animals initially perform more poorly than do conventional SPF mice in the early trials of the Barnes maze task, suggesting potential confounding effects of GF rearing on initial performance. Overall, while the data reveal an influence of the gut microbiota in modulating cognitive behavior, the complexity of the behavioral phenotypes leave open the likely possibility that there are multiple microbiota-independent effects of diet and hypoxic stress that also contribute to the observed cognitive phenotypes.

In addition to identifying a synergistic effect of KD and Hyp on impairing cognitive behavior, we also reveal an interaction between KD and Hyp in enriching Bilophila species in the gut microbiota.

While the exact mechanisms remain unknown, one study reported particular alterations in the gut microbiota that were seen when rats were exposed to both the high-fat, high-sugar diet and hypoxia in a model of obstructive sleep apnea. In a similar paradigm where atherosclerosis-prone Ldlr−/− mice were fed a high-fat diet, added exposure to hypoxia and hypercapnia increased levels of select bile acids, particularly taurodeoxycholic acid. This may be relevant, as a separate study reported that a high-saturated fat diet promoted taurine conjugation of bile acids, and that taurocholic acid in particular promoted levels of B. wadsworthia. Based on these studies and the finding that B. wadsworthia is an obligate anaerobe, we speculate that the KD and Hyp together enrich Bilophila through combined modulation of bile acid profiles and increased anaerobicity of the intestinal microenvironment.

Consistent with the finding that KD and Hyp together impair cognitive behavior and enrich Bilophila in the gut microbiota, we observe that colonization with B. wadsworthia leads to disrupted hippocampal physiology and cognitive deficits. Consistent with previous literature indicating that B. wadsworthia promotes Th1 cell expansion via IL-12p40, neutralizing IL-12p40 prevents B. wadsworthia-induced increases in intestinal Th1 cells, as well as the cognitive behavioral impairments seen in B. wadsworthia-colonized mice. Precisely how intestinal Th1 cells promote cognitive behavioral abnormalities remains poorly understood. However, increases in Th1 cells and Th1 responses are commonly associated with aging-associated cognitive decline and cognitive impairments in Alzheimer's disease. In particular, IFNy production by Th1 cells promotes microglial activation and hippocampal-dependent cognitive dysfunction.

As the prevalence of cognitive dysfunction continues to increase, identifying early and modifiable risk factors is critical to enabling early detection and intervention for cognitive impairment. Results from this study reveal that the gut microbiota is altered by modeling environmental risk factors for cognitive impairment and that changes in the gut microbiota, and Bilophila in particular, contribute to disruptions in hippocampal physiology and cognitive behavior in mice. We propose that understanding the biological bases for how complex genetic, environmental and psychosocial factors together predispose to cognitive impairment requires consideration of the gut microbiome, as an important interface between host genetics and environmental exposures and an integral regulator of host nutrition, immunity, metabolism and behavior.

### Example 2: Experimental Models and Subject Details

Mice

4-6 week old SPF wild-type Swiss Webster mice (Taconic Farms), GF wild-type Swiss Webster mice (Taconic Farms), and GF wild-type C57BL6/J mice (Jackson Laboratories) were bred in UCLA's Center for Health Sciences Barrier Facility. Mice were randomly assigned to an experimental group. Experiments include age- and sex-matched cohorts of males and females. All animal experiments were approved by the UCLA Animal Care and Use Committee.

Bacteria

B. wadsworthia was cultured under anaerobic conditions at 37° C. in Modified Brucella media (Hardy Diagnostics). C. cocleatum (DSMZ 1551) was grown in anaerobic conditions at 37° C. in Sweet E. Anaerobe Broth. Cultures were authenticated by full-length 16S rRNA sequencing.

Method Details

Dietary Treatment

Breeding GF mice were fed “breeder” chow (Lab Diets 5K52). Experimental animals were either fed standard chow (Lab Diets 5010), 6:1 ketogenic diet (Harlan Teklad TD.1150300).

Acute Intermittent Hypoxia

Hypoxia treatment was performed using an 02 Control InVivo cabinet (Coy Laboratories). For hypoxia treatment, mice were place in the chamber for 5 consecutive days for 6 hours each at an oxygen level of 12% oxygen. Mock-treated mice were in the chamber for the same amount of time at 21% oxygen.

Barnes Maze Testing

The Barnes maze was utilized as a behavioral assessment for cognitive impairment with regards to hypoxia, diet, and/or microbiome-induced changes. The following methodology was adapted from Attar et al., 2013.

The maze is from Noldus and has a navy background to capture both dark and light colored mice. The maze was made from a circular, 92 cm diameter, 5 cm hole diameter, which is mounted on a rotating stand at a height of 95 cm. The maze features 20 holes with a black escape box to make the target hole more attractive to the mice. Asymmetry of the room and simple paper color shapes (squares, triangles, circles, stars) were used as visual cues for the mice. After testing each mouse, the maze was cleaned with 70% ethanol, followed by Accel. All sessions were recorded using a Basler Gig3 camera and EthoVision XT (Noldus). Before starting testing, mice were habituated to the behavioral testing room for 1 hour at least.

The animals were tested in three phases: habituation (1 day), training (2 days), and probe trial (1 day). For the habituation day, mice were placed in a clear glass beaker in the center of the maze for 30 seconds, then they were slowly guided to the target hole and gently pushed into the escape box if they did not enter of their own accord. Mice were kept in the escape box for 1 minute and then allowed to explore the maze freely for 5 minutes, then returned to their home cages. During the training phase, mice were tested for 3 trials on the first day, and 2 trials for the second day. For each trial, mice were first placed under an opaque cup in the center of the maze for 15 seconds. Then, the cylinder was removed, and mice were allowed to explore the maze for 5 minutes. Latency to enter was defined as the time it took for mice to identify the target hole correctly for the first time. Errors made were defined as nose pokes over incorrect holes, and other metrics recorded for every trial were distance traveled, velocity, and time in each quadrant. Search strategy was also recorded, where a “random” strategy was coded as greater than 3 errors in non-consecutive holes, a “serial” strategy was coded as errors occurring in consecutive holes, an a “random” strategy was coded as greater than 3 non-consecutive hole errors. The probe trial was performed 24 hours after the final training trial. The escape box was removed, and mice were allowed to roam for 5 minutes while latency to enter, distance traveled, errors made, velocity, search strategy and time in target were recorded.

Open Field Testing

The open field task was performed in square white boxes (100 cm×100 cm). The mice were habituated in the behavioral room for an hour ahead of testing. Mice were placed in the center of the arena and behavior was monitored for 10 minutes. Mice were measured for distance traveled, time in the center versus time in the periphery of the arena, entries into the center portion, velocity, and distance traveled. The box was cleaned with 70% ethanol and Accel before each new mouse.

Pre Pulse Inhibition Testing

The following protocol is adapted from Hsiao et al 2012 and Geyer et al 1998. Mice will be acclimated to an SR-LAB testing chamber (SD Instruments) for 5 min while presented with white noise with 120-dB pulses of startle stimulus, then subjected to 14 randomized blocks of either no startle, 5-dB prepulse+startle, or 15-dB prepulse+startle. The startle response is recorded by a piezo-electric sensor and prepulse inhibition is defined as (startle stimulus only −5 or 15 dB prepulse+startle)/startle stimulus only ×100. Before and after each trial 50% Windex and dried well.

Antibiotic Treatment

SPF mice were gavaged every 12 hours daily for 7 consecutive days with a solution of vancomycin (50 mg/kg), neomycin (100 mg/kg) and metronidazole (100 mg/kg), as previously described (Reikvam et al., 2011). Ampicillin (1 mg/ml) was provided ad libitum in sterile drinking water. For mock treatment, mice were gavaged with normal drinking water every 12 hours daily for 7 days. Antibiotic-treated mice were maintained in sterile caging with sterile food and water and handled aseptically for the remainder of the experiments.

Fecal Microbiota Transplant

Fresh fecal samples were obtained from adult SPF Swiss Webster homogenized in 1 mL pre-reduced phosphate-buffered saline (PBS, pH=7.4) per pellet. 100 uL of the suspension was administered via oral gavage to recipient GF mice. For mock treatment, mice were gavaged with pre-reduced PBS.

16S rRNA Gene Sequencing

Total bacterial genomic DNA was extracted from mouse fecal samples using the Qiagen DNeasy PowerSoil Kit, where sample n reflects separate cages containing 2 mice per cage to reduce cage-dependent effects of variation and focus on biological variation. The library was prepared following methods from (Caporaso et al., 2011). The V4 regions of the 16S rDNA gene were PCR amplified using individually barcoded universal primers and 30 ng of the extracted genomic DNA. The PCR reaction was set up in triplicate, and the PCR produce was purified using the Qiaquick PCR purification kit (QIAGEN). The purified PCR product was pooled in equal molar concentrations quantified by nanodrop and sequenced by Laragen, Inc. using the Illumina MiSeq platform and 2×250 bp reagent kit for paired-end sequencing. Amplicon sequence variants (ASVs) were chosen after denoising with Deblur. Taxonomy assignment and rarefaction were performed using QIIME2-2018.6.

Gnotobiotic Colonization

10⁹ cfu bacteria were suspended in 200 ul pre-reduced PBS and orally gavaged into germ-free mice. For mock treatment, mice were gavaged with pre-reduced PBS. Mice were maintained in microisolator cages and handled aseptically. Mice were behaviorally tested 7 days post-colonization.

Hippocampal Electrophysiology

Mice were first deeply anesthetized with isoflurane; and following cervical dislocation, the brain was rapidly removed and submerged in ice-cold, oxygenated (95% O2/5% CO2) artificial cerebrospinal fluid (ACSF) containing (in mM) as follows: 124 NaCl, 4 KCl, 25 NaHCO₃, 1 NaH2PO4, 2 CaCl2, 1.2 MgSO4, and 10 glucose (Sigma-Aldrich). While iced, the brain was hemisected, and the hippocampi removed. Slice were made using a tissue chopper in 400 uM sections and maintained at 30° C. in interface-type chambers that were continuously perfused (2-3 ml/min) with ACSF and allowed to recover for at least 2 h before recordings. A bipolar nichrome wire stimulating electrode was placed in stratum radiatum of the CA1 region and used to activate Schaffer collateral fiber synapses. For LTP recordings, the maximal fEPSP amplitude was determined and the intensity of stimulation was adjusted to produce fEPSPs with an amplitude 50% of the maximal amplitude. Baseline recordings were taken for at least 20 minutes, followed by two trains of 100 Hz stimulation, then at least an hour of recording post-tetanus. The last five minutes of recording post-tetanus were used for statistical comparison. Basal synaptic strength was measure using a comparison of presynaptic fiber volleys versus fEPSP slopes. Paired-pulse facilitation was measured at impulse distances of 10, 20, 30, 40 and 50 ms.

Hippocampal Transcriptomic Profiling

Using an RNEasy Mini Kit (Qiagen), we extracted high-quality RNA (Average RIN: 8.9) as confirmed using the 4200 Tapestation (Agilent). RNA libraries were prepared using the QuantSeq FWD′ mRNA-Seq Library Prep Kit (Lexogen) and sequenced in the Illumina HiSeq platform (1×65 bp) by the UCLA Neuroscience Genomics Core. We used FastQC for quality control, followed by Trimmomatic to remove barcodes and any reads with an average phred score of 33. The following Trimmomatic parameters were also employed: illuminaclip:2:30:6, slidingwindow:5:30, leading:30, trailing:30, crop:65, minlen:20⁶³. Parsed reads were then aligned to the mouse genome mm10 using HISAT2 to identify gene identity of reads⁶⁴. We then obtained read counts using HTSeq-count⁶⁵. Differential expression of genes was determined using DESeq2 in RStudio⁶⁶. Heatmaps were constructed using the R package pheatmap, GO term enrichment analysis was conducted using DAVID, and Protein-Protein network analysis using STRING.

Hippocampal Immunofluorescence Staining and Imaging

Fixed brains were cryosectioned using a Leica CM1950 cryostat. 25 um coronal sections were collected within a span of 200 um and distributed between two slides beginning at the site of the hippocampal formation, determined in accordance to the Mouse P56 Coronal Reference Atlas of the Allen Institute. Slides were incubated in DAKO antigen retrieval solution (Agilent) at 90° C. for two minutes, washed, and then blocked (0.3% PB S-T, 5% BSA, 10% normal goat serum) for one hour at room temperature. For the examination of excitatory synapses, tissues were incubated in primary-antibody solution at 4° C. for 72 hours. After primary incubation, tissues were washed and then incubated with Alexa Fluor secondaries (1:1000) for two hours at room temperature before being washed and mounted. Primary antibody solution consisted of: anti-PSD 95 (Rabbit Polyclonal, 1:100, ThermoFisher 51-6900), anti-vGLUT 1 (Guinea Pig Polyclonal, 1:1000, Millipore AB5905), anti-vGLUT 2 (Guinea Pig Polyclonal, 1:1000, AB2251-I), anti-TuJ1 (Mouse Monoclonal, 1:200, BioLegend 801202), and anti-ZnT3 (Chicken Polyclonal, 1:500, SySy 197 006). To study inhibitory synapses and neurogenesis, tissues were incubated at 4° C. for 48 hours using the following primary antibody solution: anti-Gephyrin (Rabbit Chimeric, 1:200, SySy 147 008), anti-VGAT (Chicken Polyclonal, 1:200, SySy 131 006), and anti-DCX (Guinea Pig Polyclonal, 1:500, Millipore AB2253).

Confocal imaging for synapse analyses was performed using a Zeiss LSM 780 at 63× magnification with 1.5 zoom across 11.3 um section widths across 10 Z-stacks. Selection of synaptic puncta was strictly defined using the ImageJ plugin Puncta Analyzer with a size exclusion parameter of 0.2 um²-1.2 um² that was established by measuring co-localized puncta alongside defined axons labeled by Neuron-specific Class III B-tubulin (TuJ1)⁶⁷. DG imaging of was performed at 20× magnification with 1.5 zoom across 8.4 um section widths across 7 Z-stacks. Quantification of DCX was performed by tracing the granule cell layers of the DG and quantitating DCX+ within enclosed area using ImageJ (NIH) particle analysis. Image optimization and orthogonal projections were performed in Zen Blue (Zeiss) and background removal was done in ImageJ.

Flow Cytometry

Single-cell suspensions were prepared from lymph nodes and colonic lamina propria by enzymatic digestion or mechanical disruption. Cells were stained with fluorochrome-labelled antibodies and acquired on FACSCalibur (BD Biosciences). Data were analyzed using FlowJo (TreeStar) software.

Anti-IL-12p40 Treatment

Mice received a single bolus intraperitoneal (ip) injection of 0.5 mg of antibodies specific to p40 (C17.8, rat IgG2a) or rat isotype control antibodies (2A3, rat IgG2a) at 5 weeks of age following one week of Bilophila colonization. After the initial bolus, mice were injected ip every other day for 14 days. Both antibodies were purchased from BioXCell and diluted to 1.25 mg ml⁻¹ in PBS.

Digital PCR

Briefly, each reaction was set up with 2.0 μL of DNA sample, ddPCR master mix (QX200 ddPCR EvaGreen Supermix, #1864033, Bio-Rad Laboratories), forward (UNOOF2, 5′-CAGCMGCCGCGGTAA-3′) and reverse (UN00R0, 5′-GGACTACHVGGGTWTCTAAT-3′ [1, 3]) primers (Integrated DNA Technologies) at the final concentration of 500 nM each, and ultrapure water (Thermo Fisher Scientific) to the final volume of 20 μL. In some experiments, additional DNA intercalating dye (EvaGreen, #31000, Biotium, Fremont, Calif., USA) was added to the reactions up to ×1 final concentration (to achieve up to ×2 overall concentration). Each reaction volume was converted to droplets using a QX200 droplet generator (#1864002, Bio-Rad Laboratories). (which was not certified by peer review) is the author/funder. It is made available under a CC-BY 4.0 International license. Droplet samples were amplified on a thermocycler (C1000 Touch, #1841100, Bio-Rad Laboratories) according to the program: initial denaturation at 95° C. for 5 min. followed by 40 cycles each consisting of denaturation at 95° C. for 30 sec., annealing at 52° C. for 30 sec., and extension at 68° C. for 60 sec.; followed by the dye stabilization step consisting of 5 min incubation at 4° C., 5 min incubation at 90° C., and incubation at 12° C. for at least 5 min. Droplet samples were quantified on a QX200 Droplet Digital PCR System (#1864001, Bio-Rad Laboratories) The raw data were analyzed and the target molecule concentrations were extracted using the accompanying software (QuantaSoft Software, #1864011, Bio-Rad Laboratories).

Quantification and Statistical Analysis

Statistical analysis was performed using Prism software (GraphPad). Data were assessed for normal distribution and plotted in the figures as mean±SEM. For each figure, n=the number of independent biological replicates. No samples or animals were excluded from the analyses. Significant differences emerging from the above tests are indicated in the figures by *p<0.05, **p<0.01, ***p<0.001, ****p<0.0001. Notable non-significant (and non-near significant) differences are indicated in the figures by “n.s.”.

Data and Software Availability

16S rRNA gene sequencing data and metadata are available through QIITA repository (https://qiita.ucsd.edu/). Hippocampal transcriptomic data are available on through Gene Expression Omnibus repository.

### Example 3: The Ketogenic Diet (KD) Exacerbates Hypoxia (Hyp)-Induced Cognitive Impairment

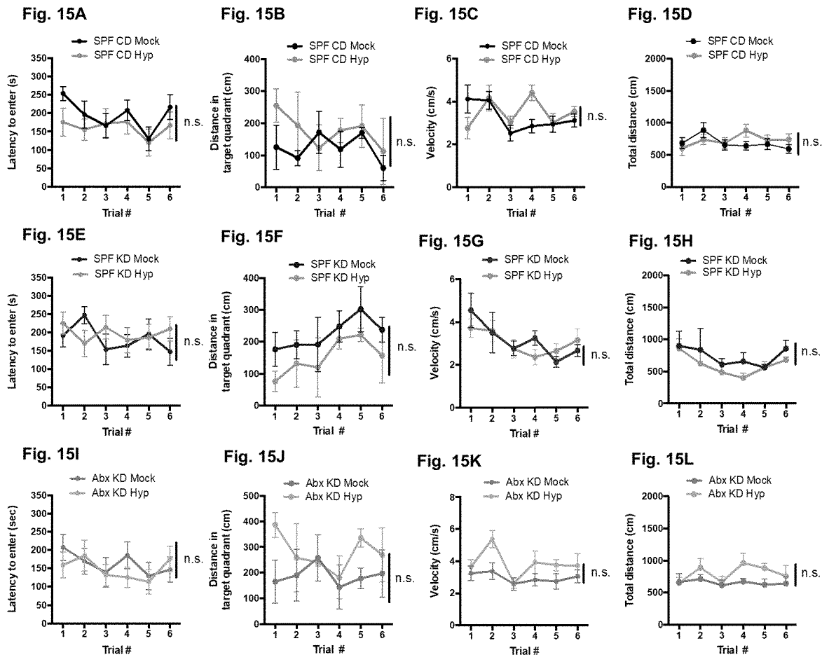

Conventional mice (specific pathogen-free, SPF) exposed to Hyp exhibited impaired cognitive behavior in the Barnes maze, which was seen as a 3.3-fold increased latency to enter the escape box, as compared to Mock controls (data not shown). This abnormality was seen in the first trial of testing and persisted through the final probe trial (Trial 6), suggesting that Hyp induced overall deficiencies in performing the cognitive task (data not shown). There was no significant difference in performance between day 1 and the probe trial in either experimental group, suggesting no evidence of learning over 6 iterations of the task (data not shown). Notably, the detrimental effect of Hyp on cognitive performance was observed in adult male mice, but not in female mice in some experiments (FIG. 15A to FIG. 15L, specifically FIG. 15A to FIG. 15D). This aligns with previous literature reporting that dihydrotestosterone exacerbates hypoxia-induced impairment in spatial memory while estrogens are protective (Zhao et al. (2004) Pediatric research 55: 498-506; Aubrecht et al. (2015) American journal of physiology. Regulatory, integrative and comparative physiology 308: R489-499; Snyder et al. (2018) Hormones and behavior 106: 62-73). Overall, these findings validate that acute intermittent hypoxia impairs cognitive performance in the Barnes maze.

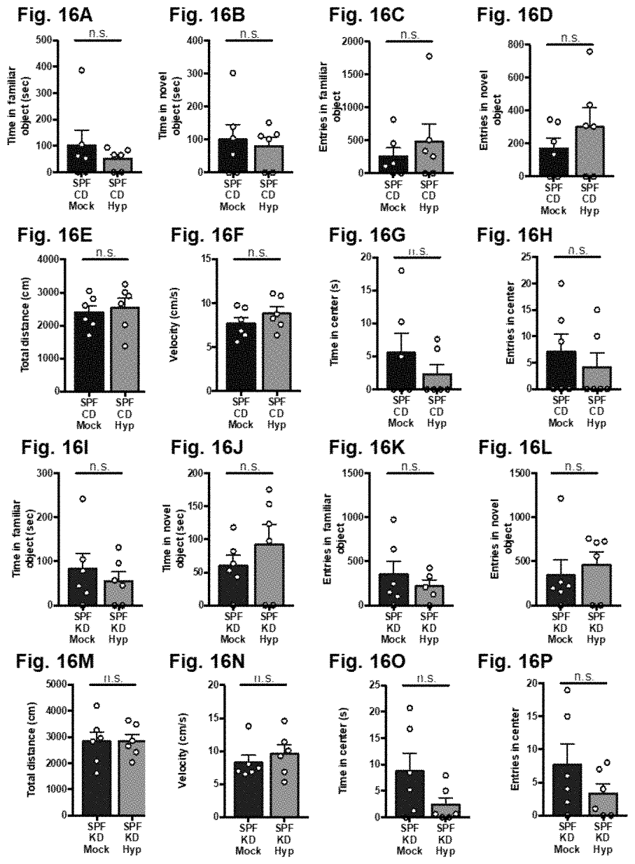

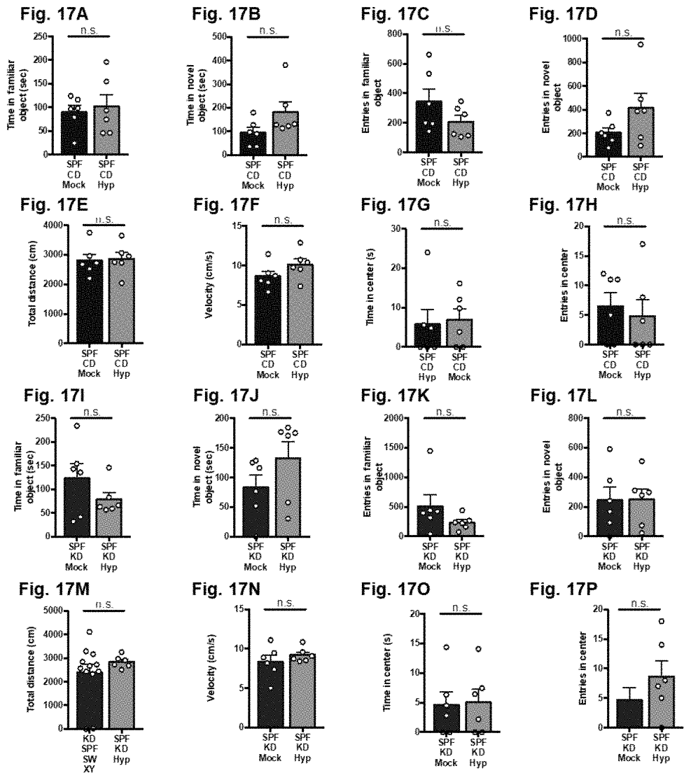

On the other hand, mice fed the high-fat, low-carbohydrate ketogenic diet (KD) and exposed to Hyp (SPF KD Hyp) exhibited a 5.1-fold increase in latency to enter the escape box as compared to Mock controls fed the KD (SPF KD Mock) (data not shown). While the overall average latency to enter decreased for SPF KD Mock mice and increased for SPF KD Hyp mice from the first to probe trial, these temporal changes were not statistically significant, suggesting a deficit in task acquisition (data not shown). When normalized to mock conditions, the SPF KD Hyp group exhibits a 5.6-fold higher response to Hyp than SPF CD mice (data not shown). This reflected a significantly higher level of Hyp-induced cognitive impairment in mice fed the KD relative to CD controls (data not shown). Consistent with behavior seen in CD-fed mice, this cognitive deficiency was seen particularly in males exposed to Hyp and not in females (FIG. 15A to FIG. 15L, specifically FIG. 15E to FIG. 1511). There was no significant difference in the velocity, errors made, or total distance traveled during the Barnes maze task (data not shown), suggesting no confounding motor abnormalities. Additionally, there was no difference across experimental groups in performance during the novel object recognition task (FIG. 16A to FIG. 16P) and the novel location recognition task (FIG. 17A to FIG. 17P), similar to cases of mild cognitive impairment (Stover et al. (2015) Behavioural brain research 289: 29-38). Altogether, these results reveal that the KD potentiates Hyp-induced cognitive impairment in the Barnes maze.

### Example 4: The Ketogenic Diet (KD) and Hypoxia (Hyp)-Associated Gut Microbiota Disrupts Cognitive Behavior

To assess whether the gut microbiota contributes to KD and Hyp-induced disruptions in Barnes maze performance, SPF mice were pre-treated with antibiotics (Abx) to deplete the gut microbiome, fed the KD, and then subjected to acute intermittent hypoxia (data not shown). Compared to vehicle-treated controls, mice that were treated with Abx were resistant to Hyp and KD-induced impairments in cognitive behavior (data not shown). In particular, Abx and Hyp-exposed mice exhibited a 2.6-fold reduction in latency to enter the escape box as compared to vehicle-treated SPF and Hyp-treated controls (data not shown). Consistent with behavior seen in SPF mice, Abx-treated mice exhibited no difference in latency to enter between the first and probe trial of the Barnes maze task despite overall decreases in latency to enter relative to SPF controls (data not shown). Notably, microbiome depletion abrogated the effects of Hyp to levels seen in CD-fed SPF mice, suggesting that the microbiome is required for the ability of KD to potentiate Hyp-induced cognitive impairment (data not shown).

To further test whether the KD and Hyp microbiota is sufficient to disrupt cognitive behavior, mice raised germ-free (GF) were transplanted with fecal microbiota from SPF mice exposed to KD and Hyp, or to Mock controls (GF+SPF KD Hyp and GF+SPF KD Mock respectively) (data not shown) (Mohle et al. (2016) Cell reports 15: 1945-1956; Hueston et al. (2017) Translational psychiatry 7: e1081). Mice colonized with SPF KD Hyp microbiota exhibited poor cognitive performance, akin to that seen in SPF mice exposed to KD and Hyp, whereas mice that received SPF KD Mock transplants exhibited comparatively better performance in the Barnes maze task (data not shown). Specifically, mice colonized with the Hyp microbiota displayed a 1.3-fold higher latency to enter relative to mice colonized with the Mock microbiota (data not shown). Interestingly, the Mock microbiota was not sufficient to confer the level of cognitive ability seen in native SPF mice exposed to KD and Mock (data not shown). This suggests that adult conventionalization of GF mice only partially improves cognitive performance. (data not shown). Indeed, colonization with SPF KD Mock microbiota as measured by a significant reduction in latency to enter in the probe trial compared to trial 1, while SPF KD Hyp microbiota colonization produced no significant latency difference (data not shown). Consistent with the ability of the KD and Hyp microbiota to confer impairment, there was no evidence of learning for mice colonized with the SPF KD Hyp microbiota (data not shown). When normalized to mock conditions, the SPF KD Hyp group exhibits a 5.6-fold higher response to Hyp than SPF CD mice that is significantly reduced by either antibiotic pretreatment (Abx KD) or germ-free rearing (GF (data not shown). Recolonization with the SPF KD Hyp microbiota increases the hypoxia effect size and sufficiently confers the Hyp-associated cognitive impairment (data not shown). Together, these results suggest that the KD and Hyp-associated microbiota disrupts cognition.

### INCORPORATION BY REFERENCE

Each publication and patent mentioned herein is hereby incorporated by reference in its entirety. In case of conflict, the present specification, including any definitions herein, will control.

### EQUIVALENTS

While specific embodiments of the subject invention have been discussed, the above specification is illustrative and not restrictive. Many variations of the invention will become apparent to those skilled in the art upon review of the preceding description and the following claims. The full scope of the invention should be determined by reference to the claims, along with their full scope of equivalents, and by reference to the rest of the specification, along with such variations.

What is claimed is:

1. A microbiome modulator for use in treatment of hypoxia-induced cognitive impairment in a subject.

2. The microbiome modulator for use according to claim 1, wherein the microbiome modulator comprises a ketogenic-diet-suppressed bacterial species.

3. The microbiome modulator for use according to claim 2, wherein the bacterial species is Clostridium cocleatum.

4. The microbiome modulator for use according to claim 1, wherein the microbiome modulator comprises an antimicrobial agent active against a ketogenic-diet-boosted bacterial species.

5. The microbiome modulator for use according to claim 4, wherein the antimicrobial agent is an antibiotic effective against Bilophila wadsworthia.

6. The microbiome modulator for use according to claim 5, wherein the antibiotic is imipenem, cefoxitin, or ticarcillin.

7. The microbiome modulator for use according to any one of claims 4 to 6, wherein the microbiome modulator further comprises Clostridium cocleatum.

8. A method of selecting a subject having hypoxia-induced cognitive impairment, the method comprising
obtaining from a sample of a subject a test level for a biomarker associated with hypoxia-induced cognitive impairment; and
selecting the subject if the test level differs from a control level for said biomarker by more than a predetermined threshold.

9. The method of claim 8, wherein the control level is representative of a level of said biomarker in a subject that does not have hypoxia-induced cognitive impairment.

10. The method of claim 8 or 9, wherein the biomarker comprises an RNA or a polypeptide of a gene selected from Actb, Atg2a, Atp5d, Atp6v0e2, Camkv, Cldn11, Cldn5, Dctn4, Erbb3, Gabarap, Mag, Mapk11, Mbp, Micall1, Mobp, Nfasc, Nipal4, Pik3r2, Scn1b, Tubd1, and Zfpm1, wherein the test level is lower than the control level, and wherein the predetermined threshold is 20% of the control level.

11. The method of claim 8 or 9, wherein the biomarker comprises an RNA or a polypeptide of a gene selected from Adam7, Adcyap1, Adig, Adipoq, Adora2a, Adrb3, Aoc3, Avp, Baiap3, C3, Calb2, Car3, Cartpt, Cbin1, Cdo1, Ceacam10, Cidec, Cwc22, Defb20, Defb48, Dio2, Drd1, Ecel1, Etnppl, Fabp4, Fgf12, Fggy, Flvcr2, G0s2, Gad2, Glra1, Glra3, Gm42743, Gm44862, Gpx5, Hp, K1h11, Lcn8, Lgr5, Lyzf1, Marcks, mCG_18947, Meis1, Myo19, Pbx3, Penk, Plin1, Plin4, Pmch, Pnpla2, Prdm1, Retn, Retnla, Rgs9, Rrad, Rspo1, Scd1, Spink3, Spink1, Sslp1, Svs1, Svs4, Svs5, Svs6, Tacr1, and Wisp1, wherein the test level is higher than the control level, and wherein the predetermined threshold is 20% of the control level.

12. A method of treating hypoxia-induced cognitive impairment in a subject, the method comprising administering an effective amount of a microbiome modulator to the subject.

13. The method of claim 12, wherein the microbiome modulator comprises a ketogenic-diet-suppressed bacterial species.

14. The method of claim 13, wherein the bacterial species is Clostridium cocleatum.

15. The method of claim 12, wherein the microbiome modulator comprises an antimicrobial agent active against a ketogenic-diet-boosted bacterial species.

16. The method of claim 15, wherein the antimicrobial agent is an antibiotic effective against Bilophila wadsworthia.

17. The method of claim 16, wherein the antibiotic is imipenem, cefoxitin, or ticarcillin.

18. The method of any one of claims 15 to 17, further comprising administering an effective amount of Clostridium cocleatum.

19. A method of treating a subject having hypoxia-induced cognitive impairment, the method comprising
selecting a subject having hypoxia-induced cognitive impairment in whom the test level of at least one biomarker associated with hypoxia-induced cognitive impairment differs from a control level for said biomarker by more than a predetermined threshold; and
administering an effective amount of a microbiome modulator to the subject.

20. The method of claim 19, wherein the control level is representative of a level of said biomarker in a subject that does not have hypoxia-induced cognitive impairment.

21. The method of claim 19 or 20, wherein the biomarker comprises an RNA or a polypeptide of a gene selected from Actb, Atg2a, Atp5d, Atp6v0e2, Camkv, Cldn11, Cldn5, Dctn4, Erbb3, Gabarap, Mag, Mapk11, Mbp, Micall1, Mobp, Nfasc, Nipa14, Pik3r2, Scn1b, Tubd1, and Zfpm1, wherein the test level is lower than the control level, and wherein the predetermined threshold is 20% of the control level.

22. The method of claim 19 or 20, wherein the biomarker comprises an RNA or a polypeptide of a gene selected from Adam7, Adcyap1, Adig, Adipoq, Adora2a, Adrb3, Aoc3, Avp, Baiap3, C3, Calb2, Car3, Cartpt, Cbin1, Cdo1, Ceacam10, Cidec, Cwc22, Defb20, Defb48, Dio2, Drd1, Ecel1, Etnppl, Fabp4, Fgf12, Fggy, Flvcr2, G0s2, Gad2, Glra1, Glra3, Gm42743, Gm44862, Gpx5, Hp, Klhl1, Lcn8, Lgr5, Lyzf1, Marcks, mCG_18947, Meis1, Myo19, Pbx3, Penk, Plin1, Plin4, Pmch, Pnpla2, Prdm1, Retn, Retnla, Rgs9, Rrad, Rspo1, Scd1, Spink3, Spink1, Sslp1, Svs1, Svs4, Svs5, Svs6, Tacr1, and Wisp1, wherein the test level is higher than the control level, and wherein the predetermined threshold is 20% of the control level.

23. The method of any one of claims 19 to 22, wherein the microbiome modulator comprises Clostridium cocleatum.

24. The method of any one of claims 19 to 23, wherein the microbiome modulator comprises a beta-lactam antibiotic.

25. A method of obtaining a prognostic indicator of hypoxia-induced cognitive impairment in a subject who receives a dosage of a microbiome modulator, the method comprising
obtaining from a sample of a subject after administration of a test dose of the microbiome modulator to the subject a test level for a biomarker associated with hypoxia-induced cognitive impairment;
determining that the test level differs from a reference level for said biomarker by a test-reference differential by comparing the test level with said reference level; and
determining that the hypoxia-induced cognitive impairment of the subject is improving if the test-reference differential is less than a predetermined differential.

26. The method of claim 25, wherein the reference level is a control level that is representative of a level of said biomarker in a subject that does not have hypoxia-induced cognitive impairment.

27. The method of claim 26, wherein the predetermined differential is a predetermined threshold equal to 20% of the reference level.

28. The method of claim 25, wherein the reference level is obtained from a sample of the subject before the administration of a test dose of the microbiome modulator to the subject.

29. The method of claim 28, wherein the predetermined differential is equal to 10% of the reference level.

30. The method of any one of claims 25 to 29, further comprising decreasing the dosage of the microbiome modulator.

31. The method of any one of claims 25 to 30, wherein the biomarker comprises an RNA or a polypeptide of a gene selected from Actb, Atg2a, Atp5d, Atp6v0e2, Camkv, Cldn11, Cldn5, Dctn4, Erbb3, Gabarap, Mag, Mapk11, Mbp, Micall1, Mobp, Nfasc, Nipa14, Pik3r2, Scnlb, Tubd1, Zfpm1, Adam7, Adcyap1, Adig, Adipoq, Adora2a, Adrb3, Aoc3, Avp, Baiap3, C3, Calb2, Car3, Cartpt, Cbin1, Cdo1, Ceacam10, Cidec, Cwc22, Defb20, Defb48, Dio2, Drd1, Ecel1, Etnppl, Fabp4, Fgf12, Fggy, Flvcr2, G0s2, Gad2, Glra1, Glra3, Gm42743, Gm44862, Gpx5, Hp, Klhl1, Lcn8, Lgr5, Lyzf1, Marcks, mCG_18947, Meis1, Myo19, Pbx3, Penk, Plin1, Plin4, Pmch, Pnpla2, Prdm1, Retn, Retnla, Rgs9, Rrad, Rspo1, Scd1, Spink3, Spink1, Sslp1, Svs1, Svs4, Svs5, Svs6, Tacr1, and Wisp1.

32. The microbiome modulator for use or the method of any one of claims 3, 7, 14, 18, and 23, wherein the microbiome modulator comprises Clostridium cocleatum at an amount between 100 million and 20 billion colony forming units.

33. The microbiome modulator for use or the method of any one of claims 1 to 32, wherein the subject is a male human.

34. A method of treating hypoxia-induced cognitive impairment in a subject, the method comprising administering an effective amount of an anti-IL-12p40 agent to the subject.

35. The method of claim 34, wherein the anti-IL-12p40 agent comprises an anti-IL-12p40 antibody or an antigen-binding fragment thereof.
